(19) United States
(12) Patent Application Publication
(10) Pub. No.: US 2011/0021560 A1
(43) Pub. Date: Jan. 27, 2011

(54) Compositions And Methods For Regulating Chondrocyte Proliferation In Bone Disorders

(75) Inventors: Paul Joseph Fanning, Groton, MA (US); John J. Wixted, Northborough, MA (US)

Correspondence Address: MEDLEN & CARROLL, LLP, 101 HOWARD STREET, SUITE 350, SAN FRANCISCO, CA, 94105, US

(73) Assignee: University of Massachusetts Medical School

(21) Appl. No.: 12/821,586

(22) Filed: Jun. 23, 2010

Related U.S. Application Data
(60) Provisional application No. 61/219,719, filed on Jun. 23, 2009.

(51) Int. Cl. A61K 31/47; A61P 19/00

(52) U.S. Cl. 514/311

(57) ABSTRACT

The present invention is related to the field of cartilage physiology, repair, and regeneration. In particular, the invention contemplates a treatment for bone healing disorders, especially those related to the articular joints and bone, by upregulating chondrocyte proliferation. For example, inhibition of cysteinyl leukotriene activity on chondrocytes by using cysteinyl leukotriene-1 receptor antagonists may be useful in preventing and treating bone healing disorders. This invention also relates to other physiologic conditions which are influenced by chondrocyte activity, including pediatric long bone growth and neoplastic conditions involving cells of chondrogenic origin

### FIELD OF THE INVENTION

The present invention is related to the field of cartilage physiology, repair, and regeneration. In particular, the invention contemplates a treatment for bone healing disorders, especially those related to the articular joints and bone, by upregulating chondrocyte proliferation. For example, inhibition of cysteinyl leukotriene activity on chondrocytes by using cysteinyl leukotriene-1 receptor antagonists may be useful in preventing and treating bone healing disorders. This invention also relates to other physiologic conditions which are influenced by chondrocyte activity, including pediatric long bone growth and neoplastic conditions involving cells of chondrogenic origin

### BACKGROUND

The costs of treatment for traumatic injuries represent a significant biomedical burden. For example, the 2002 US Health Cost & Utilization Project reported that hospital costs for cranial surgery (craniotomies and craniectomies) and facial trauma reconstruction alone were estimated to be approximately $549 million and $400 million, respectively. Steiner et al., Eff Clin Pract, 2002. 5(3): p. 143-51). The hospital costs for orthopedic surgeries (both trauma and nontrauma) are likely even higher as the figure for orthopedic industry sales alone was estimated to be $13 billion in 2002 (Medical Technology Fundamentals, Merrill Lynch, 2003. p. 11).

Overall, the major problem encountered in the treatment of traumatic injuries of the axial skeleton concern the modulation of cartilage and/or bone formation. Preferably, cartilage formation can be increased under conditions in which it would be desirable to have more or accelerated bone formation as part of the treatment of certain conditions (e.g., orthopedic or craniofacial fracture repair, spinal fusion surgery, joint fusion surgery, injured osteoporotic bone) or as part of the prevention of certain conditions (e.g., fracture prevention in osteoporotic bone). The process by which long bones heal is termed endochondral ossification; this is a highly regulated and sequential process of cellular differentiation and matrix deposition which requires primary chondrogenesis for it progress to later stages of bone formation. For successful long bone fracture repair, it may be desirable to have accelerated endochondral ossification by upregulating chondrogenesis; Forming a large and rapid cartilage anlage would eventually be followed, in later stages of healing, by immature and later mature bone formation. Even more preferably, endochondral ossification can also be decreased under conditions in which it would be desirable to have decreased or inhibited bone formation as part of the treatment or prevention of certain conditions (e.g., craniosynostosis, a condition of premature calvarial overgrowth across sutures leading to premature suture fusion; heterotopic ossification, a condition of abnormal bone formation in ectopic locations). Similarly, it would be preferred to increase cartilage formation under conditions in which it would be desirable to have more or accelerated cartilage formation (e.g., joint resurfacing, temporomandibular joint reconstruction, articular disc repair, intervertebral disc repair and regeneration).

Nonetheless, there is no curative treatment for lost bone mass associated with bone healing disorders, including various growth-promoting proteins and Vitamin D3. Likewise, there is no effective replacement or implant for some bone healing disorders, such as non-union fractures or crush injuries of the bone. Currently, these latter types of injury utilize a variety of synthetic and/or allograft human bone tissue which is chemically treated to remove proteins in order to prevent rejection. However, such bone substitutes, while mechanically important, are biologically dead and do not contain bone-forming cells, growth factors, or other regulatory proteins. Thus, they are not capable of modulating the repair process.

Currently, there are no methods available to regulate chondrogenesis. As the growth and differentiation of chondrocytes is the initial step in endochondral ossification, regulating the process may be highly beneficial in treating a variety of traumatic conditions of the appendicular skeleton. For these reasons, compositions and methods for regulating the growth and activity of chondrocytes are needed for the treatment of injuries of the joints and bone may also be applied to neoplastic processes involving cells of chondrogenic origin.

### SUMMARY OF THE INVENTION

The present invention is related to the field of cartilage physiology, repair, and regeneration. In particular, the invention contemplates a treatment for bone healing disorders, especially those related to the articular joints and bone, by upregulating chondrocyte proliferation. For example, inhibition of cysteinyl leukotriene activity on chondrocytes by using cysteinyl leukotriene-1 receptor antagonists may be useful in preventing and treating bone healing disorders. This invention also relates to other physiologic conditions which are influenced by chondrocyte activity, including pediatric long bone growth and neoplastic conditions involving cells of chondrogenic origin

In one embodiment, the present invention contemplates a method, comprising: a) providing; i) a patient comprising at least one symptom of a bone healing disorder; ii) a composition comprising a cysteinyl-leukotriene receptor antagonist capable of reducing the symptom; b) administering the receptor antagonist to the patient under conditions such that the symptom is reduced. In one embodiment, the bone healing disorder is selected from the group including, but not limited to, non-union predisposition, non-healing non-union fractures, osteopenia, osteogenesis imperfecta, critical size defects, non-critical size defects, osteochondral defects, subchondral defects, endochondromas, chondrosarcomas, or osteochondritis dessicans. In one embodiment, the patient further comprises a chondrocyte, wherein the chondrocyte expresses at least one cysteinyl leukotriene-1 (CysLT1) receptor. In one embodiment, the administering of the receptor antagonist stimulates the chondrocyte to proliferate. In one embodiment, the receptor antagonist comprises montelukast. In one embodiment, the receptor antagonist comprises a montelukast derivative. In one embodiment, the receptor antagonist comprises a peptide. In one embodiment, the receptor antagonist comprises an oligonucleotide. In one embodiment, the oligonucleotide comprises an antisense oligonucleotide. In one embodiment, the administering is parenteral. In one embodiment, the administering is oral. In one embodiment, the administering is intraarticular. In one embodiment, the bone disorder is caused by a disease. In one embodiment, the bone disorder is congenital.

### DEFINITIONS

The term “a bone healing disorder” as used herein, refers to bone defects including, but not limited to, non-union predisposition, non-union fractures, osteopenia, osteogenesis imperfecta, critical size defects, non-critical size defects, osteochondral defects, subchondral defects, endochondromas, chondrosarcomas, and defects resulting from degenerative diseases such as osteochondritis dessicans. In one embodiment, the present invention contemplates a method for treating and/or repairing non-healing, non-union defects and for promoting articular cartilage repair in chondral or osteochondral defects.

The term “accelerated” as used herein, refers to chondrocyte-mediated osteogenesis occurring more rapidly as compared to a bone healing disease. Such rapid osteogenesis may be a direct result of chondrocyte proliferation such that a bone grows more quickly in a treated subject as compared to an untreated subject or a control subject.

The term “enhanced” or “enhancing” as used herein, refers to a treated bone in a patient having a bone healing disorder, having improved characteristics as compared to an untreated subject, or a control subject such as, for example, greater bone strength.

The term “fracture healing” or “fracture repair” as used herein, refers to promoting the healing of bone fractures and bone defects, and improving the mechanical stability of the healing fracture or site. Such bone fractures may include, but are not limited to; i) trauma-induced non-osteoporotic fractures; ii) osteoporotic fractures due to osteoporosis or osteopenia of any etiology; iii) fractures due to Paget's disease; iv) fractures due to bone loss as a consequence of side effects of other drugs, e.g. in patients receiving high doses of corticosteroids; v) fractures arising from congenital bone healing disorders such as, e.g., osteogenesis imperfecta; vi) surgical created fractures (i.e., for example, osteotomies) used for example in bone lengthening and limb lengthening procedures; and vii) treatment of bone fracture delayed unions or non-unions.

The term “osteogenesis” as used herein, refers to any production of bone. For example, such bone production may be associated with repair of a bone that has a defect caused by a bone healing disorder, intentional or non-intentional damage, or induction of bone formation used to fuse more than one bone or bone segment together. For example, one method to induce osteogenesis comprises chondrocyte proliferation.

The term “bone defect” as used herein, refers to any abnormality of a bone such that a portion of the bone is missing. For example, a bone defects includes, but is not limited to, anomalous holes, gaps or -A openings created in the bone for purposes of a diagnostic or therapeutic procedure, loss of bone segments from trauma or disease, puncture wounds to the bone, and the like.

The term “bone formation” as used herein, refers the generation of new bone in a subject treated according to the methods of the invention, such as, e.g., by receiving a CysLT1 antagonist, that is increased over bone generation in a subject that is not given a CysLT1 antagonist. Bone formation may be determined by method such as quantitative digitized morphometry, as well as by other markers of bone formation. Bone formation is meant to include the osteogenic process used for spine fusions and other joint or bone ankylosis application, bone formation into or around prosthetic devices, or bone formation to augment existing bones or replace missing bones or bone segments. Bone formation may also means formation of endochondral bone or formation of intramembranous bone. In humans, bone formation begins during the first 6-8 weeks of fetal development. Progenitor stem cells of mesenchymal origin migrate to predetermined sites, where they either: (a) condense, proliferate, and differentiate into bone-forming cells (osteoblasts), a process observed in the skull and referred to as “intramembranous bone formation;” or, (b) condense, proliferate and differentiate into cartilage-forming cells (chondroblasts) as intermediates, which are subsequently replaced with bone-forming cells. More specifically, mesenchymal stem cells differentiate into chondrocytes. The chondrocytes then become calcified, undergo hypertrophy and are replaced by newly formed bone made by differentiated osteoblasts, which now are present at the site. Subsequently, the mineralized bone is extensively remodeled, thereafter becoming occupied by an ossicle filled with functional bone-marrow elements. This process is observed in long bones and referred to as “endochondral bone formation.” In postfetal life, bone has the capacity to repair itself upon injury by mimicking the cellular process of embryonic endochondral bone development. That is, mesenchymal progenitor stem cells from the bone-marrow, periosteum, and muscle can be induced to migrate to the defect site and begin the cascade of events described above. There, they accumulate, proliferate, and differentiate into cartilage, which is subsequently replaced with newly formed bone.

The term “bone” as used herein, refers to any calcified (mineralized) connective tissue primarily comprising a composite of deposited calcium and phosphate in the form of hydroxyapatite, collagen (primarily Type I collagen) and bone cells such as chondrocytes, osteoblasts, osteocytes, and osteoclasts, as well as to bone marrow tissue which forms in the interior of true endochondral bone. Bone tissue differs significantly from other tissues, including cartilage tissue. Specifically, bone tissue is a vascularized tissue comprising cells and a biphasic medium comprising a mineralized, inorganic component (primarily hydroxyapatite crystals) and an organic component (primarily of Type I collagen). Glycosaminoglycans constitute less than 2% of this organic component and less than 1% of the biphasic medium itself, or of bone tissue per se. Moreover, relative to cartilage tissue, the collagen present in bone tissue exists in a highly-organized parallel arrangement. Bony defects, whether from degenerative, traumatic or cancerous etiologies, pose a formidable challenge to the reconstructive surgeon. Particularly difficult is reconstruction or repair of skeletal parts that comprise part of a multi-tissue complex, such as occurs in mammalian joints.

The term “cartilage formation” as used herein, means formation of connective tissue containing chondrocytes embedded in an extracellular network comprising fibrils of collagen (predominantly Type II collagen along with other minor types such as Types IX and XI), various proteoglycans, other proteins and water. “Articular cartilage” refers specifically to hyaline or articular cartilage, an avascular non-mineralized tissue which covers the articulating surfaces of the portions of bones in joints and allows movement in joints without direct bone-to-bone contact, thereby preventing wearing down and damage of opposing bone surfaces. Normal healthy articular cartilage is referred to as “hyaline,” i.e. having a characteristic frosted glass appearance. Under physiological conditions, articular cartilage tissue rests on the underlying, mineralized bone surface called subchondral bone, which contains highly vascularized ossicles. The articular, or hyaline cartilage, found at the end of articulating bones is a specialized, histologically distinct tissue and is responsible for the distribution of load resistance to compressive forces, and the smooth gliding that is part of joint function. Articular cartilage has little or no self-regenerative properties. Thus, if the articular cartilage is torn or worn down in thickness or is otherwise damaged as a function of time, disease or trauma, its ability to protect the underlying bone surface is comprised. In normal articular cartilage, a balance exists between synthesis and destruction of the above-described extracellular network. Other types of cartilage in skeletal joints include fibrocartilage and elastic cartilage. Secondary cartilaginous joints are formed by discs of fibrocartilage that join vertebrae in the vertebral column. In fibrocartilage, the mucopolysaccharide network is interlaced with prominent collagen bundles and the chondrocytes are more widely scattered than in hyaline cartilage. Elastic cartilage contains collagen fibers that are histologically similar to elastin fibers. Cartilage tissue, including articular cartilage, unlike other connective tissues, lacks blood vessels, nerves, lymphatics and basement membrane. Cartilage is composed of chondrocytes, which synthesize an abundant extracellular milieu composed of water, collagens, proteoglycans and noncollagenous proteins and lipids. Collagen serves to trap proteoglycans and to provide tensile strength to the tissue. Type II collagen is the predominant collagen in cartilage tissue. The proteoglycans are composed of a variable number of glycosaminoglycan chains, keratin sulphate, chondroitin sulphate and/or dermatan sulphate, and N-lined and O-linked oligosaccharides covalently bound to a protein core.

The term “articular” or “hyaline” cartilage as used herein, can be distinguished from other forms of cartilage by both its morphology and its biochemistry. Certain collagens such as the fibrotic cartilaginous tissues, which occur in scar tissue, for example, are keloid and typical of scar-type tissue, i.e., composed of capillaries and abundant, irregular, disorganized bundles of Type I and Type II collagen. In contrast, articular cartilage is morphologically characterized by superficial versus mid versus deep zones which show a characteristic gradation of features from the surface of the tissue to the base of the tissue adjacent to the bone. In the superficial zone, for example, chondrocytes are flattened and lie parallel to the surface embedded in an extracellular network that contains tangentially arranged collagen and few proteoglycans. In the mid zone, chondrocytes are spherical and surrounded by an extracellular network rich in proteoglycans and obliquely organized collagen fibers. In the deep zone, close to the bone, the collagen fibers are vertically oriented. The keratin sulphate rich proteoglycans increase in concentration with increasing distance from the cartilage surface. For a detailed description of articular cartilage microstructure, see, for example, (Aydelotte and Kuettner, (1988), Conn. Tiss. Res. 18:205; Zanetti et al., (1985), J. Cell Biol. 101:53; and Poole et al., (1984), J. Anat. 138:13. Biochemically, articular collagen can be identified by the presence of Type II and Type IX collagen, as well as by the presence of well-characterized proteoglycans, and by the absence of Type X collagen, which is associated with endochondral bone formation.

The term “articular defect” as used herein refers to articular surface defects, i.e., full-thickness defects and superficial defects. These defects differ not only in the extent of physical damage to the cartilage, but also in the nature of the repair response each type of lesion can elicit. Full-thickness defects, also referred to herein as “osteochondral defects,” of an articulating surface include damage to the hyaline cartilage, the calcified cartilage layer and the subchondral bone tissue with its blood vessels and bone marrow. Full-thickness defects can cause severe pain, since the bone plate contains sensory nerve endings. Such defects generally arise from severe trauma. Full-thickness defects may, on occasion, lead to bleeding and the induction of a repair reaction from the subchondral bone. In such instances, however, the repair tissue formed is a vascularized fibrous type of cartilage with insufficient biomechanical properties, and does not persist on a long-term basis. In contrast, superficial defects in the articular cartilage tissue are restricted to the cartilage tissue itself. Such defects, also referred to herein as “chondral” or “subchondral defects”, are notorious because they do not heal and show no propensity for repair reactions. Superficial defects may appear as fissures, divots, or clefts in the surface of the cartilage. They contain no bleeding vessels (blood spots), such as those seen in full-thickness defects. Superficial defects may have no known cause, but they are often the result of mechanical derangements that lead to a wearing down of the cartilaginous tissue. Such mechanical derangements may be caused by trauma to the joint, e.g., a displacement of torn meniscus tissue into the joint, meniscectomy, a taxation of the joint by a torn ligament, malalignment of joints, or bone fracture, or by hereditary diseases. Since the cartilage tissue is not innervated or vascularized, superficial defects do not heal and often degenerate into full-thickness defects.

The term “defect” or “defect site”, as used herein, can define a bony structural disruption requiring repair. The defect further can define an osteochondral defect, including a structural disruption of both the bone and overlying cartilage. A defect can assume the configuration of a “void”, which is understood to mean a three-dimensional defect such as, for example, a gap, cavity, hole or other substantial disruption in the structural integrity of a bone or joint. A defect can be the result of accident, disease, surgical manipulation, and/or prosthetic failure. In certain embodiments, the defect is a void having a volume incapable of endogenous or spontaneous repair. Such defects are generally twice the diameter of the subject bone and are also called “critical size” defects. For example, in a canine ulna defect model, the art recognizes such defects to be approximately 34 cm, generally at least approximately 2.5 cm, gap incapable of spontaneous repair. See, for example, Schmitz et al., Clinical Orthopaedics and Related Research 205:299-308 (1986); and Vukicevic et al., in Advanced in Molecular and Cell Biology, Vol. 6, pp. 207-224 (1993) (JAI Press, Inc.), the disclosures of which are incorporated by reference herein. In rabbit and monkey segmental defect models, the gap is approximately 1.5 cm and 2.0 cm, respectively. In other embodiments, the defect is a non-critical size segmental defect. Generally, these are capable of some spontaneous repair, albeit biomechanically inferior to those made possible by practice of the instant innovation. In certain other embodiments, the defect is an osteochondral defect, such as an osteochondral plug. Such a defect traverses the entirety of the overlying cartilage and enters, at least in part, the underlying bony structure. In contrast, a chondral or subchondral defect traverses the overlying cartilage, in part or in whole, respectively, but does not involve the underlying bone. Other defects susceptible to repair using the instant invention include, but are not limited to, non-union fractures; bone cavities; tumor resection; fresh fractures (distracted or undistracted); cranial/facial abnormalities; periodontal defects and irregularities; spinal fusions; as well as those defects resulting from diseases such as cancer, and other bone degenerative disorders such as osteochondritis dessicans.

The term “repair” as used herein, refers to any new bone and/or cartilage formation which is sufficient to at least partially fill the void or structural discontinuity at the defect. Repair does not, however, mean, or otherwise necessitate, a process of complete healing or a treatment which is 100% effective at restoring a defect to its pre-defect physiological/structural/mechanical state.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc) to a binding partner (i.e., for example, a CysLT1 receptor) under conditions such that the binding partner becomes unresponsive to its natural ligands. Inhibitory compounds may include, but are not limited to, small organic molecules, antibodies, proteins/peptides, and oligonucleotides such as antisense oligonucleotides.

The term “cysteinyl-leukotriene receptor” as used herein, refers to any protein capable of binding a cysteinyl-leukotriene compound. For example, a cysteinyl-leukotriene receptor may reside in the cell membrane and respond to circulating levels of cysteinyl-leukotrienes in order to mediate various physiological responses. The type of response depends upon cysteinyl-leukotriene receptor subtype (i.e., for example, CysLT1 or CysLT2).

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “medium” as used herein, refers to any material, or combination of materials, which serve as a carrier or vehicle for delivering of a drug to a treatment point (e.g., wound, surgical site etc.). For all practical purposes, therefore, the term “medium” is considered synonymous with the term “carrier”. It should be recognized by those having skill in the art that a medium comprises a carrier, wherein said carrier is attached to a drug or drug and said medium facilitates delivery of said carrier to a treatment point. Further, a carrier may comprise an attached drug wherein said carrier facilitates delivery of said drug to a treatment point. Preferably, a medium is selected from the group including, but not limited to, foams, gels (including, but not limited to, hydrogels), xerogels, microparticles (i.e., microspheres, liposomes, microcapsules etc.), bioadhesives, or liquids. Specifically contemplated by the present invention is a medium comprising combinations of microparticles with hydrogels, bioadhesives, foams or liquids. Preferably, hydrogels, bioadhesives and foams comprise any one, or a combination of, polymers contemplated herein. Any medium contemplated by this invention may comprise a controlled release formulation. For example, in some cases a medium constitutes a drug delivery system that provides a controlled and sustained release of drugs over a period of time lasting approximately from 1 Day to 6 months.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering” a drug or compound, as used herein, refers to any method of providing a drug or compound to a patient such that the drug or compound has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “patient”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions.

The term “binding” as used herein, refers to any interaction between an infection control composition and a surface. Such as surface is defined as a “binding surface”. Binding may be reversible or irreversible. Such binding may be, but is not limited to, non-covalent binding, covalent bonding, ionic bonding, Van de Waal forces or friction, and the like. An infection control composition is bound to a surface if it is impregnated, incorporated, coated, in suspension with, in solution with, mixed with, etc.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents an illustration of one embodiment of a physiological lipid metabolic pathway.

FIG. 2 presents the chemical structure of montelukast.

FIG. 3 presents exemplary radiographs of a mouse femur bone fracture model taken a Day 0. A: Montelukast sodium group. B: Zileuton Group. C: Control group.

FIG. 4 presents exemplary histological data showing that treatment with either montelukast sodium or zileuton enhances early stages of fracture repair. Animals were sacrificed at Days 7, 10, and 14. Histologic specimens were stained with Safranin-O to detect cartilage. (photographed at 40×).

FIG. 5A presents exemplary data showing total cartilage area based upon the measurements of the histological data presented in FIG. 4.

FIG. 5B presents exemplary data showing percent of total callous area based upon the measurements of FIG. 5A.

FIG. 6 presents exemplary data showing the effect of montelukast sodium and zileuton for CysLT1 receptor mRNA expression (A) and 5-LO mRNA expression (B) at Day 7, 10, 14, and 21 following mouse model bone fracture initiation.

FIG. 7A presents a schematic diagram showing one embodiment of a fracture callous region.

FIG. 7B presents exemplary data showing an immunohistochemical analysis of chondrocyte BLT1 receptors in fracture callous expressing LTB4. day 7 staining is primarily cytoplasmic, and by day 10 staining is both cytoplasmic and nuclear.

FIG. 8 presents exemplary photomicrographs (100×) showing abundant collagen 2 staining that confirms the presence of chondroid matrix at days 7, 10, and 14. Expression of CysLT1 and 5-LO are present in this chondroid tissue.

FIG. 9 presents exemplary micrographs of the data in FIG. 8 at higher a magnification (200×). CysLT1 receptor expression is clearly seen in a restricted chondrocyte population, with no staining in fibroblast appearing cells. A similar pattern in seen with 5-LO expression. Negative controls confirm specificity of staining.

FIG. 10 presents exemplary data showing that cysteinyl leukotrienes are negative regulators of chondrocyte proliferation. Col 2a1 mRNA expression (A) and SOX9 mRNA expression (B) in fracture callous tissue is shown at days 7, 10, 14, and 21 after administering montelukast sodium or zileuton.

FIG. 11 presents exemplary data showing that administration of montelukast sodium and zileuton leads to enhanced chondrocyte hypertrophy and early bone formation. Runx2 mRNA expression (A) and Col 10a1 mRNA expression (B) were analyzed as markers of hypertrophic chondrocyte formation and compared to osteocalcin mRNA expression (C).

### DETAILED DESCRIPTION

The present invention is related to the field of cartilage physiology, repair, and regeneration. In particular, the invention contemplates a treatment for bone healing disorders, especially those related to the articular joints and bone, by upregulating chondrocyte proliferation. For example, inhibition of cysteinyl leukotriene activity on chondrocytes by using cysteinyl leukotriene-1 receptor antagonists may be useful in preventing and treating bone healing disorders. This invention also relates to other physiologic conditions which are influenced by chondrocyte activity, including pediatric long bone growth and neoplastic conditions involving cells of chondrogenic origin

### I. Cysteinyl-Leukotriene Pathway

Leukotrienes (LTs), a family of lipid mediators, has long been reported to play a role in the pathogenesis of inflammation. For example, LTB4 is believed to exert it action through G protein coupled receptors, LTB4 R-1 and LTB4 R-2. On the other hand, cysteinyl-leukotrienes are believed to be bronchoconstrictors, thereby mediating asthmatic conditions. Such observations has lead to speculation that LTs, in general, may mediate inflammatory response and LT modifying agents including zileuton, zafirlukast and montelukast may be useful to treat inflammatory conditions.

The importance of the early inflammatory response is widely recognized, and many reports over several decades have examined the effects of commonly used non-steroidal anti-inflammatory drugs (NSAIDs) on many conditions including, but not limited to, bone fracture repair. These drugs, which modulate the initial inflammatory response, can have long-term effects on the later stages of fracture repair. (Anon., 1978; Sudmann et al., 1979; Keller et al., 1989; Engesaeter et al., 1992; Hogevold et al., 1992; Beck et al., 2003; Bergenstock et al., 2005; Murnaghan et al., 2006; Gerstenfeld et al., 2007; Simon and O'Connor, 2007). The immediate physiologic response to skeletal injury is a local and systemic inflammatory reaction. This first stage of fracture repair is followed by subsequent stages which culminate in skeletal regeneration. This cascade of events results in mesenchymal stem cell (MSC) recruitment to the zone of injury and terminal stem cell differentiation. Chondrocyte proliferation and differentiation predominate early in the process, giving way to matrix resorption, osteoblastic differentiation, and osseous tissue formation. In this sense, the skeletal repair process is recognized as highly sequential, progressing in a step wise fashion through well recognized stages (Schindeler et al., 2008). The early stages of repair are critical for successful healing, and inflammatory mediators which drive the initial cellular response set in motion a complex interplay of chondrogenesis, osteogenesis, and neovascularization (Einhorn, 2005).

Taken collectively, these studies demonstrated that arachidonic acid metabolism to prostaglandins may be important for healing, and that inhibiting prostaglandin synthesis by administration of cyclo-oxygenase inhibitors generally results in delayed skeletal repair. Arachidonic acid is formed from cell membrane bound phospholipids in response to many biological signals including injury. Collectively, the biologically relevant metabolites of arachidonic acid are known as eicosanoids, and comprise the families of prostaglandins (PGs), thromboxanes (TXs), leukotrienes (LTs), and lipoxins (LXs). The eicosanoids provide a wide range of inflammatory effects, variously affecting mucous membranes, airway constriction, gastric acid secretion, pain and fever modulation, and affect initiation of labor, among others (Boyce, 2008). Currently, there is limited knowledge concerning a potential role for leukotrienes and their cognate receptors in modulating fracture repair, particularly in early chondrogenic phases. One preliminary report using 5-lipoxygenase (5-LO) knockout mice demonstrated larger callous size and enhanced mechanical properties after fracture, although the mechanism for this effect remained unclear (Manigrasso and O'Connor, 2006). While the effects of prostaglandins on fracture repair have been well studied, the effects of other ecoisanoid families on skeletal regeneration have not received the same attention.

5-LO functions for the leukotriene family in a manner analogous to cyclo-oxygenase in the prostaglandin family. This enzyme, acting in concert with 5-lipoxygenase activating protein (FLAP), catalyzes the conversion of arachidonic acid first to a series of intermediary metabolites, with the end result formation of two major groups of leukotrienes. These two groups comprise LTB4, and what is collectively known as the cysteinyl leukotrienes including, but not limited to, LTC4, LTD4, LTE4, or LTF4.

The cysteinyl leukotrienes, as a group, have previously been implicated as negative regulators of MSC differentiation (Akino et al., 2006). In vitro studies of human MSCs cultured in the presence of pranlukast, a specific cysteinyl leukotriene type 1 (CysLT1) receptor antagonist, showed enhanced cellular differentiation based on morphology, highlighting the potential inhibitory effect of the CysLT1 pathway on MSC differentiation. Thus, it is possible that MSC differentiation could be promoted during fracture repair via CysLT1 receptor inhibition. While it has long been recognized that local tissue mechanical trauma causes an up regulation of leukotriene production, the effect of these potent inflammatory mediators on fracture repairs remains unknown. (Denzlinger et al., 1985)

For example, the cysteinyl-leukotrienes are believed to exert a range of proinflammatory effects, such as constriction of airways and vascular smooth muscle, increase of endothelial cell permeability leading to plasma exudation and edema, and enhanced mucus secretion. Cysteinyl-leukotrienes have been reported to mediate asthma, allergic rhinitis, and other inflammatory conditions, including cardiovascular diseases, cancer, atopic dermatitis, and urticaria.

Subtyping of cysteinyl-LT receptors (CysLTRs) was based initially on binding and functional data, obtained using natural agonists and a wide range of antagonists. CysLTRs have proved remarkably resistant to cloning. However, in 1999 and 2000, the CysLT1R and CysLT2R were successfully cloned and both shown to be members of the G-protein coupled receptors (GPCRs) superfamily. Molecular cloning has confirmed most of the previous pharmacological characterization and identified distinct expression patterns that are only partially overlapping. Recombinant CysLTRs couple to the Gq/11 pathway that modulates inositol phospholipid hydrolysis and calcium mobilization, whereas in native systems, they often activate a pertussis toxin-insensitive G_{i/o}-protein, or are coupled promiscuously to both G-proteins. Recent data provide evidence for the existence of an additional receptor subtype that seems to respond to both cysteinyl-LTs and uracil nucleosides, and of an intracellular pool of CysLTRs that may have roles different from those of plasma membrane receptors. An interaction between cysteinyl-LT and the purine systems has also been hypothesized. Rovati et al., “Cysteinyl-leukotriene receptors and cellular signals” Scientific World Journal 7:1375-1392 (2007).

The cysteinyl leukotrienes (CysLTs) may include, but are not limited to, LTC(4), LTD(4) or LTE(4), and are believed to trigger contractile and inflammatory processes through the specific interaction with cell surface receptors. It is further believed that these CysLT cell surface receptors may belong to a purine receptor cluster of the rhodopsin family of the G protein-coupled receptor (GPCR) genes. Pharmacological studies have identified at least two classes of CysLT receptors (i.e., for example, CysLT(1) or CysLT(2)) based on their sensitivity to CysLT(1) selective antagonists, and there is evidence for additional subtypes. Molecular cloning of the human CysLT(1) and CysLT(2) receptors has confirmed both their structure as putative seven transmembrane domain G protein-coupled receptors and most of the previous pharmacological characterization. The rank order of potency of agonist activation for the CysLT(1) receptor is LTD4>LTC4>LTE4 and for the CysLT(2) receptor is LTC4=LTD4>LTE4. The CysLT(1) receptor is most highly expressed in spleen, peripheral blood leukocytes, interstitial lung macrophages and in airway smooth muscle. The CysLT(2) receptor is mostly expressed in heart, adrenals, placenta, spleen, peripheral blood leukocytes and less strongly in the brain. Capra V., “Molecular and functional aspects of human cysteinyl leukotriene receptors” Pharmacol Res. 50:1-11 (2004).

Given the evidence that the CysLT1 receptor acts as a negative regulator of MSC differentiation (Akino et al., 2006), a process involved in fracture repair, along with previous murine fracture studies indicating larger callous size and enhanced mechanical properties of healing fractures in a 5-LO knockout model, mediators of leukotriene synthesis may be used to enhance normal, uncomplicated fracture repair. (Manigrasso and O'Connor, 2006).

In one embodiment, the present invention contemplates specifically enhancing chondrogenesis in clinical situations involving severely disordered healing of the joints or bone, as in the situation of nonunion, delayed union, or injury to the articular surface of a joint. In one embodiment, chondrogenesis enhancements improves the healing and treatment of the underlying disorder when normal healing is likely to be disrupted.

### II. Cysteinyl-Leukotriene Receptor Antagonists (LTRAs)

Cysteinyl-leukotriene receptor antagonists (LTRAs) were introduced as oral preventative anti-asthma medications in the late 1990s and, very recently, montelukast has been approved by the United States Food and Drug Agency for the relief of symptoms of perennial and seasonal allergic rhinitis. Although clinical trials and clinical practice showed LTRAs to be effective in the treatment of asthma, their exact role in the therapy of asthma is not well defined and possibly under-appreciated. Clinical trials with LTRAs uncovered a range of patient responses, so that an understanding of the variability mechanisms (e.g. acquired or genetic factors, etc.) is needed to maximize the probability of a beneficial response. Since the molecular cloning of CysLT receptors (CysLTRs) has been achieved, new roles for cysteinyl-LTs in pathophysiological conditions have been suggested or established from the observed distribution in cells and tissues other than the lung. Capra et al., “Cysteinyl-leukotriene receptor antagonists: present situation and future opportunities” Curr Med. Chem. 13:3213-3226 (2006).

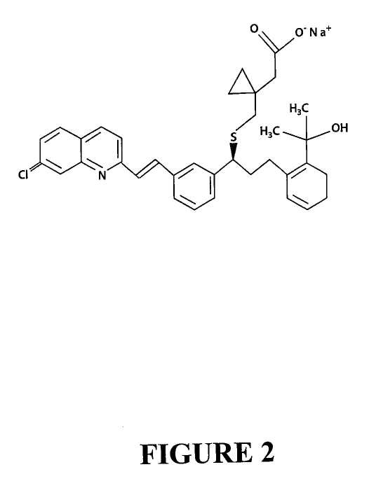

Specifically, clinically used LTRAs include, but are not limited to, montelukast (Singulair®: [1-[[1-[3-[2-[(7-chloro-2-quinolyl)]vinyl]phenyl]-3-[2-(1-hydroxy-1-methyl-1-ethyl)phenyl]-propyl]sulfanylmethyl]-cyclopropyl]acetic acid) (See, FIG. 2), zafirlukast (Accolate®: 3-[[2-methoxy4-(o-tolylsulfonylcarbamoyl)-phenyl]methyl]-1-methyl-1H-indol-5-yl]aminoformic acid cyclopentyl ester) or pranlukast (Onon®: 4-oxo-8-[p-(4-phenylbutyloxy)benzoylamino]-2-tetrazol-5-yl)-4H-1-benzopyr-an), MCC-847 (ZD-3523), MN-001, MEN-91507 (LM-1507), VUF-5078, VUF-K-8707, L-733321 and 1-(((R)-(3-(2-(6,7-difluoro-2-quinolinyl)ethenyl)phenyl)-3-(2-(2-hydroxy-2-propyl)phenyl)thio)methyl cyclopropane-acetic acid, 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneac-etic acid [2-[[2-(4-tert-butyl-2-thiazolyl)-5-benzofuranyl]oxymethyl]pheny-1]acetic acid optionally in the form of the racemates, enantiomers or diastereomers thereof and optionally in the form of the pharmacologically acceptable acid addition salts, solvates and/or hydrates thereof. Further, acid addition salts of the betamimetics may be selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which LTRAs may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates, or furoates.

Montelukast, montelukast derivatives, and compatible formulations have been reported as a putative LTRA family. Tung et al., “Process For 3-(2-(7-Chloro-2-Quinolinyl)ethenyl)-benzaldehyde” U.S. Pat. No. 5,869,673 (Issued: Feb. 9, 1999); Hagmann et al., “Substituted Aminoquinolines As Modulators Of Chemokine Receptor Activity” U.S. Pat. No. 5,919,776 (Issued: Jul. 6, 1999); and Arison et al., “Quinoline Leukotriene Antagonists” U.S. Pat. No. 5,952,347 (Issued: Sep. 14, 1999) (all patents herein incorporated by reference).

Montelukast sodium is a selective and orally active leukotriene receptor antagonist that inhibits the cysteinyl leukotriene-1 receptor. It is useful as an anti-asthmatic, anti-allergic, anti-inflammatory and/or cytoprotective agent. Montelukast sodium is indicated for the prophylaxis and chronic treatment of asthma in adults and pediatric patients 6 years of age and older. The dosage for adolescents and adults 15 years of age and older is typically one 10-mg tablet daily to be taken in the evening.

Montelukast sodium is a hygroscopic white to off-white powder, which is freely soluble in ethanol, methanol, and water and practically insoluble in acetonitrile. One reported synthesis of montelukast sodium proceeds through a corresponding methyl ester. EP 480717. The methyl ester of montelukast is hydrolyzed to the free acid that can later be converted directly to the corresponding sodium salt. This process is not particularly suitable for large-scale production because it requires tedious chromatographic purification of the methyl ester intermediate and/or the final product, and the product yields are low.

An improved process for the preparation of crystalline montelukast sodium salt, which comprises the generation of a dilithium dianion of 1-(mercaptomethyl)cyclopropaneacetic acid followed by condensation with 2-(2-(3(S)-(3-(2-(7-chloro-2-quinolinyl)ethenyl)phenyl)-3-methanesulfonyl-oxypropyl)phenyl)-2-propanol to afford the montelukast acid which is then converted, via the dicyclohexyl amine salt of montelukast to its corresponding sodium salt. The obtained sodium salt is further crystallized from a mixture of toluene:acetonitrile to obtain crystalline montelukast sodium. WO 95/18107.

A montelukast dicyclohexyl amine salt is reported to be a useful intermediate for the purification of the crude montelukast acid before its conversion to the desired sodium salt. In the crystalline state, the montelukast dicyclohexylamine salt was obtained in two polymorphic modifications.

One process for preparation of montelukast sodium has been reported comprising: (i) providing a solution of starting montelukast free acid in a halogenated solvent, aromatic solvent, or mixtures thereof; (ii) treating said solution with a source of sodium ion to convert said montelukast free acid into a sodium salt of montelukast; (iii) adding a cyclic or acyclic hydrocarbon solvent to said solution thereby precipitating said sodium salt of montelukast. US Application Publication No. 2005/0107612. Montelukast acids may also be generated in situ from an amine salt of montelukast, whereby specifically mentioned amines include tert-butylamine and phenyl ethylamine. US Application Publication No. 2005-0234241. Montelukast purification may also proceed via its dicyclohexylamine salt. WO/2004-108679. Several forms of montelukast free acid and a process for preparing montelukast free acid. The process generally includes a step of liberating montelukast free acid from its salt. One of the specifically mentioned salts (and the only amine salt) is dicyclohexylamine salt. WO 2005/074935.

Amantadine salts of montelukast have also been reported. Amantadine, or more properly 1-aminoadamantane, is a known pharmaceutical active ingredient and a salt formed with montelukast is thus pharmaceutically acceptable. The salt can be in solid state, especially a crystalline form or state. The crystalline montelukast salt can have a high purity, such as when used as an intermediate for purifying and/or isolating montelukast acid or if used as an active agent directly, including purities of at least 95% and preferably at least 98%. Pharmaceutical compositions comprising an amantadine salt of montelukast have been adapted for oral or nasal inhalation. Such solutions may contain montelukast, amantadine, and/or their ions in a solvent; wherein an amantadine salt of montelukast is crystallized from the solution. Bartl et al., “Montelukast Amantadine Salt” U.S. Pat. No. 7,446,116.

### III. Bone Healing Disorders

In one embodiment, the present invention contemplates a method for preventing and/or treating the initiation and/or development of bone healing disorders including, but not limited to, a non-union bone disorder. For example, risk factors for developing a non-union bone disorder includes but is not limited to, diabetes, smoking, obesity, and ‘at risk’ surgical patient individuals due to impaired blood flow to their extremities.

In one embodiment, a bone healing disorder may also be characterized by bone defects including, but not limited to, non-union predisposition, non-union fractures, osteopenia, osteogenesis imperfecta, critical size defects, non-critical size defects, osteochondral defects, subchondral defects, and defects resulting from degenerative diseases such as osteochondritis dessicans. In one embodiment, the present invention contemplates a method for treating and/or repairing non-healing, non-union defects and for promoting articular cartilage repair in chondral or osteochondral defects.

Bone non-union disorders have been suggested to be mediated by predispositional factors. For example, treatment of diaphyseal nonunion of long bones is difficult and controversial. A retrospective review of 113 patients with diaphyseal nonunion treated by various modalities identified 36 cases of nonunion of the tibia, 23 nonunions of the femur, 21 nonunions of the humerus, 13 nonunions of the radius, 18 nonunions of the ulna and two nonunions of the clavicle. These nonunions were classified as aseptic (84) and septic (29) and additionally classified as hypertrophic (61) and atrophic (52) in order to determine the treatment. While all fractures eventually healed, residual problems were seen in some patients including, but not limited to, joint stiffness, limb length discrepancy, and angular deformity. Furthermore, twenty-six patients required repeat surgery using bone grafting because no satisfactory progress of fracture healing was seen in 4 months. Some complications were observed believed related to the iliac crest donor site and persistent infection at the nonunion site. Babhulkar et al., “Nonunion of the diaphysis of long bones” Clin Orthop Relat Res. 431:50-56 (2005). Another study suggested that problems related to consolidation may be linked with an overall reduction of bone marrow progenitor cells, as a result of some general physiological problems (i.e., for example, chemotherapy, smoking, alcoholic poisoning). Hernigou et al., “Pseudarthrosis treated by percutaneous autologous bone marrow graft” Rev Chir Orthop Reparatrice Appar Mot. 83:495-504 (1997). This study characterized the bone marrow from 35 non-union sites, not only with respect to the medullary stroma but also the hematopoietic compartment. The in vitro activity of bone marrow was compared between nonunion sites with that of samples taken from the iliac crest. The non-union cases included, post-traumatic non union, prosthetic arthrodesis non-union, tibiotarsal arthrodesis non-union, non-regenerated illizarov extensions, and congenital abnormalities. The data showed that non union sites and extension regenerated fibrous tissue had relatively few F-CFU to differentiate into fibroblasts. In 12 out of 35 patients studied, the bone marrow generated no F-CFU, whereas these same patients have abnormal low levels of F-CFU obtainable from their iliac crest bone marrow. The number of GM-CFU in fracture site is also extremely low. It has been reported that non-union disorders are commonly expressed in traumatic segmental femoral fractures requiring immediate surgery. Reconstructive procedures are often delayed due to the priority for repair of soft tissue wounds. Consequently, the complication rate is high and femoral non-unions are not uncommon. However, spontaneous unions have been observed in some patients while waiting for a definitive skeletal reconstructive procedure. This has been suggested to be due to a genetic predispostion for enhanced bone repair. Hinsche et al. “Spontaneous healing of large femoral cortical bone defects: does genetic predisposition play a role?” Acta Orthop Belg. 69:441-446 (2003).

Osteopenia is characterized by an unexplained decrease in the amount of calcium and phosphorus in the bone. This can cause bones to be weak and brittle, and increases the risk for broken bones. During the last 3 months of pregnancy, large amounts of calcium and phosphorus are transferred from the mother to the baby so that the baby's bones will grow. A premature infant may not receive the proper amount of calcium and phosphorus needed to form strong bones. While in the womb, fetal activity increases during the last 3 months of pregnancy. This activity is thought to be important for bone development. Most very premature infants have limited physical activity, which may also contribute to weak bones. Very premature babies lose much more phosphorus in their urine than do babies that are born full term. A lack of vitamin D may also lead to osteopenia in infants. Vitamin D helps with the body absorb calcium from the intestines and kidneys. If babies do not receive or make enough vitamin D, calcium and phosphorous will not be properly absorbed. A liver problem called cholestasis may also cause problems with vitamin D levels. Diuretics or steroids can also cause low calcium levels. Most premature infants born before 30 weeks have some degree of osteopenia, but will not have any physical symptoms. Infants with severe osteopenia may have decreased movement or swelling of an arm or leg due to an unknown fracture. Osteopenia is more difficult to diagnose in premature infants than in adults. The most common tests used to diagnose and monitor osteopenia of prematurity include: Blood tests to check levels of calcium, phosphorus, and a protein called alkaline phosphatase, ultrasound, or X-rays. Therapies currently being used include: calcium, phosphorus, or vitamin D supplementation.

Osteogenesis imperfecta is also a condition causing extremely fragile bones. Osteogenesis imperfecta (OI) is a congenital disease, meaning it is present at birth. It is frequently caused by defect in the gene that produces type 1 collagen, an important building block of bone. There are many different defects that can affect this gene. The severity of OI depends on the specific gene defect. OI is an autosomal dominant disease. Most cases of OI are inherited from a parent, although some cases are the result of new genetic mutations. Symptoms are reflected in the fact that all people with OI have weak bones, which makes them susceptible to fractures. Persons with OI are usually below average height (short stature). However, the severity of the disease varies greatly. In general symptoms include: blue tint to the whites of their eyes (blue sclera), multiple bone fractures, or hearing loss including deafness. Because type I collagen is also found in ligaments, persons with OI often have loose joints (hypermobility) and flat feet. Some types of OI also lead to the development of poor teeth. More severe symptoms may include: bowed legs and arms, kyphosis, scoliosis (S-curve spine).

Size defects (i.e., for example, critical and/or non-critical) may be related to commonly observed inefficient healing of bony and cartilaginous defects. It has been suggested that such defects may be treated by enhancing the regenerative potential of bone and articular cartilage having the potential for treatment of nonunions, large segmental bone and cartilage defects. Issack et al., “Recent advances toward the clinical application of bone morphogenetic proteins in bone and cartilage repair” Am J. Orthop. 32:429-436 (2003). Further, others have reported a gene therapy approach to treat skeletal defects for the purpose of promoting bone repair. For example, fractures at risk for delayed unions or nonunions. Animal models including rats, dogs, and sheep, showed that the delivery of plasmids for parathyroid hormone or bone morphogenetic protein promoted bone formation and the healing of critical size defects. Goldstein S. A., “In vivo nonviral delivery factors to enhance bone repair” Clin Orthop Relat Res. 379(Suppl):S113-S119 (2000). One report suggests that in some cases, injuries where there is an underlying bone disorder will not heal spontaneously unless technology is used. For such bone disorders include, but are not limited to, normal fracture healing, the segmental loss of bone or critical size defects, and various forms of nonunions in which fracture healing is perturbed either by mechanical, metabolic, or neurologic means. Einhorn T. A., “Clinically applied models of bone regeneration in tissue engineering research” Clin Orthop Relat Res. 367 Suppl:S59-S67 (1999).

Osetochondral defects may involve articular cartilage that is often damaged due to trauma or degenerative diseases, resulting in severe pain and disability. Most clinical approaches have been shown to have limited capacity to treat cartilage lesions. Tissue engineering (TE) has been proposed as an alternative strategy to repair cartilage. Cartilage defects often penetrate to the subchondral bone, or full-thickness defects are also produced in some therapeutic procedures. Mano et al., “Osteochondral defects: present situation and tissue engineering approaches” J Tissue Eng Regen Med. 1:261-273 (2007). Osteochondral defects may also include, anterior ankle problems, such as anterior impingement syndrome, that are commonly treated using arthroscopic surgery. Niek van Dijk et al., “Advancements in ankle arthroscopy” J Am Acad Orthop Surg. 16:635-46 (2008). Damaged articular cartilage has a limited capacity for self-repair, and is also usually treated with conventional surgical techniques. Some suggested treatments involving implantation of autologous chondrocytes in suspension or within a variety of cell carrying scaffolds such as hyaluronic acid, alginate, agarose/alginate, fibrin or collagen. For the repair of full-thickness osteochondral defects, a single- or bi-phased scaffold constructs often contain hydroxyapatite-collagen composites, usually used as a bone substitute. Chajra et al., “Collagen-based biomaterials and cartilage engineering. Application to osteochondral defects” Biomed Mater Eng; 18(1 Suppl):S33-S45 (2008).

A subchondral defect may include, but is not limited to, a carpal bone defect involving the scaphoid, lunatum, and hamatum. Bilateral defects may be observed. Different mechanisms have been put forward to explain the development of intraosseous defects in the carpal bones including intraosseous penetration of synovial tissue, or in situ metaplasia of bone tissue. The main differential diagnoses are osteonecrosis sequellae (for the lunatum and the scaphoid), subchondral defects due to hyperpression and arthropathies in dialysis patients. Masmejean et al., “Primary carpal bone defect” Rev Chir Orthop Reparatrice Appar Mot. 86:80-86 (2000). Polymethylmethacrylate (PMMA) is often used to fill the large subchondral defects following intralesional curettage of a giant cell tumor of the bone. While Steinmann pins have been used to reinforce the bone cement, it is controversial as to whether this procedure has any real benefit. Asavamongkolkul et al., “Stability of subchondral bone defect reconstruction at distal femur: comparison between polymethylmethacrylate alone and steinmann pin reinforcement of polymethylmethacrylate” J Med Assoc Thai. 86:626-633 (2003). The etiology and pathophysiology of Perthes' disease have remained elusive and treatment is controversial. Arthrography has demonstrated a fluid-filled space between the ossified epiphysis of the femoral head and its overlying articular cartilage. Such a finding in this mechanically vulnerable region suggests that this region may be subject to mechanical distortion, thereby contributing to a primary symptom in Perthes' disease; a femoral head deformity. Knight et al., “Arthrographically defined subchondral defects in Perthes' disease” J Pediatr Orthop B. 17:73-76 (2008).

Endochondromas are bone healing disorders that have a characteristic appearance of a tumor, including, but not limited to subungual verrucae, endochondroma, fibroma, or amelanotic melanoma. For example, a subungual exostosis arises underneath the nail plate, originating from the underlying bone. With such a wide variety of similar-appearing tumors, optimal treatment of this disorder presently is limited to proper recognition and treatment. Woo et al., “Subungual osteocartilaginous exostosis” J Dermatol Surg Oncol. 11:534-536 (1985).

A “chondroma and chondrosarcoma” are bone healing disorders characterized as a chondrogenetic benign or malignant tumor. A “chondroma” comprises a benign tumor generated from mesodermal cells to be differentiated into cartilage, and includes enchondroma generated from medullary cavity, extraskeletal chondroma generated in soft tissue and having no connection with the bone or periosteum beneath the tissue, periosteal chondroma generated from periosteum or connective tissue of periosteum, parosteal chondroma, etc. A chondrosarcoma” comprises a malignant tumor originating from cartilage cells, and includes central chondrosarcoma generated in the central part of the bone, peripheral chondrosarcoma generated from the cartilage cap of osteochondroma, chondrosarcoma derived from undifferentiated cells of mesenchymal origin having cartilage differentiation ability, etc. Some chondrosarcomas shift from chondromas and therefore the boundary therebetween is sometimes not clear, and for this reason, they are collectively referred to as “chondroma and chondrosarcoma” in this specification.

Osteochondrosis is a common and clinically important joint disorder that occurs in human beings and in multiple animal species, most commonly pigs, horses, and dogs. This disorder is defined as a focal disturbance of enchondral ossification and is regarded as having a multifactorial etiology, with no single factor accounting for all aspects of the disease. The most commonly cited etiologic factors are heredity, rapid growth, anatomic conformation, trauma, and dietary imbalances; however, only heredity and anatomic conformation are well supported by the scientific literature. The way in which the disease is initiated has been debated. Although formation of a fragile cartilage, failure of chondrocyte differentiation, subchondral bone necrosis, and failure of blood supply to the growth cartilage all have been proposed as the initial step in the pathogenesis, the recent literature strongly supports failure of blood supply to growth cartilage as being the most likely. The term osteochondrosis has been used to describe a wide range of different lesions among different species. Refinements of this disease may include, but not limited to, the modifiers latens (lesion confined to epiphyseal cartilage), manifesta (lesion accompanied by delay in endochondral ossification), and dissecans (cleft formation through articular cartilage). Ytrehus et al. “Etiology and pathogenesis of osteochondrosis” Vet Pathol. 44:429-448 (2007). Osteochondritis dissecans (OCD) and subchondral bone cysts (SBCs) occur commonly and at many different locations in limbs. Depending on the location and extent of the lesion, arthroscopic surgical debridement may be an effective treatment. In many cases, however, additional techniques to improve the healing response in bone and cartilage are needed so as to preserve articular function. Methods for improving cartilage repair (i.e., restoration of damaged cartilage) or regeneration (i.e., reformation or recreation of new articular cartilage) are suggested. Fortier et al., “New surgical treatments for osteochondritis dissecans and subchondral bone cysts” Vet Clin North Am Equine Pract. 21:673-690 (2005).

It is clear that research into pre-existing bone healing disorders (i.e., for example, a non-union bone healing disorder) has not identified effective preventive and/or therapeutic strategies. In the normal physiologic state, it is believed that fracture healing progresses through a sequential cascade of inflammation, chondrogenesis, followed by calcified cartilage formation, and in later stages by replacement of the calcified cartilage anlage with first immature woven bone, and over a prolonged period remodeling to mature bone. In the pathologic case of atrophic non-union, for example, this sequence is interrupted and normal healing does not occur.

In one embodiment, the present invention contemplates a method for inhibiting a chondrocyte CysLT1 receptor following oral administration of a specific CysLT1 receptor antagonist under conditions such that the symptoms of a bone healing disorder are reduced. Although it is not necessary to understand the mechanism of an invention, it is believed that given the sequential nature of the healing process, bone cannot be created by endochondral ossification unless cartilage is formed first; thus, enhancing chondrogenesis may address the high propensity for disordered healing seen in patients with diabetes, patients who take medications which interfere with fracture healing, patients who smoke, patients with underlying vasculopathy, or patients with other identified risk factors for non-union. It is further believed that the data presented herein provide evidence that oral leukotriene inhibitors promote bone healing at early chondrogenic stages, thereby accelerating and enhancing endochondral bone formation suggests that this treatment may also be effective in treating and prevention pre-existing bone healing disorders. In particular, the data presented herein demonstrates, for the first time, that chondrocytes express an CysLT1 receptor.

### IV. Traumatic Bone Fracture Healing

Trauma-induced fracture repair occurs in stages including, but not limited to; i) inflammation; ii) regeneration; or iii) remodeling. Skeletal injury is believed to initiate a cascade of events that results in mesenchymal stem cell recruitment to the zone of injury and terminal differentiation to a chondroid lineage (i.e., for example, resulting in the production of chondrocytes). Some small organic molecules (i.e., for example, drugs) may modulate this initial response that can result in downstream effects on the much later (i.e., subsequent) stages of fracture repair. It is believed that skeletal repair processes may proceed as highly sequential and progress in a step-wise fashion. Thus, it is further believed that early stages of repair play a role in successful bone healing, and early inflammatory mediators (which have the initial cellular response) set in motion a complex interplay of chondrogenesis, osteogenesis, and neovascularization.

The early inflammatory response occurring after bone fracture, has resulted in the common use of non-steroidal anti-inflammatory drugs (NSAIDs). However, some studies have shown that arachidonic acid metabolism and resultant prostaglandin production may play a positive role in bone healing. For example, inhibiting arachidonic acid metabolism by administering cyclo-oxygenase inhibitors generally results in delayed skeletal repair. Clinical correlations are less clear than animals models, but a number of investigators have reported negative effects on skeletal regeneration following the use of NSAIDs in humans. (supra) Fracture healing is a complex tissue regeneration process that involves cell migration, proliferation, apoptosis, and differentiation in response to growth factors, cytokines, other signaling molecules, and to the mechanical environment. The temporal order and magnitude of each cellular process must be controlled for optimal regeneration. The normal events of fracture healing are described below as occurring in 4 phases.

In the initial phase, hematoma formation and localized tissue hypoxia are the initial cellular and molecular events of fracture healing. The second phase, called the early stage, is characterized by inflammation followed by rapid accumulation of cells at the fracture site. The presence of macrophages and neutrophils at the fracture site during inflammation precedes the rapid migration and proliferation of mesenchymal cells at the fracture site. In the third, regenerative phase, endochondral ossification creates the new bone which bridges the fracture. At this point, the fracture callus has a well-defined morphology. Intramembraneous ossification creates buttresses of veriosteal bone at the callus periphery. Mesenchymal cells within the callus begin to differentiate into chondrocytes at the interface of the veriosteal bone buttress. Each new chondrocyte develops as would be expected with matrix deposition followed by matrix calcification to produce calcified cartilage and then apoptosis. Channels are formed into the calcified cartilage starting at the periosteal bone buttresses. Osteoblasts migrate or differentiate on the surface of the calcified cartilage within these channels and begin depositing new bone. As chondrocyte differentiation proceeds from the periphery to the center of the callus (fracture site), channel formation, osteoblast differentiation, and new bone formation follows until the soft callus has been replaced with woven (immature) bone. Angiogenesis during the regenerative phase also occurs.

The immature woven bone created during the regenerative phase is mechanically unsuited for normal weight-bearing. To compensate for the decreased mechanical properties of the woven bone, the fracture callus has a significantly larger diameter which provides for greater structural mechanical properties. In the final, remodeling phase, fracture callus diameter diminishes until the bone obtains its normal dimensions while maintaining the bones overall mechanical properties by enhancing material mechanical properties. This is accomplished by replacing the mechanically poor, woven bone with mechanically strong, lamellar (mature) bone. In successive rounds, osteoclasts resorb the woven bone and osteoblasts replace it with lamellar bone. Molecular mechanisms governing osteoclast formation and function occurs through the RANKL-RANK pathway and this pathway is activated during fracture healing.

### V. LTRA Treatment of Bone Healing Disorders

One convenient model to predict potential therapies for bone healing disorders involve bone fracture animal models. While treating bone healing disorders and treating trauma-induced bone fractures may appear similar, the data presented herein shows that one having ordinary skill in the art could not have predicted the involvement of chondrocyte CysLT1 receptors in treating bone healing disorders by the state of the art for reducing inflammatory responses following trauma-induced bone healing. Consequently, the data discussed below in the context of bone fracture healing is not to be interpreted as limiting. Discussion of bone fracture healing data presented herein is merely illustrative in order to represent some embodiments described herein relative to treating bone healing disorders that do not yet have art accepted research models.

In one embodiment, the present invention contemplates a method for activating the proliferation of chondrocytes by inhibiting the interaction of cysteinyl leukotrienes with a CysLT1 receptor. The data presented herein demonstrate that chondrocyte CysLT1 receptors represent a potential medical strategy to prevent and treat the initiation and/or development of bone healing disorders including, but not limited to, a non-union bone disorder. For example, risk factors for developing a non-union bone disorder includes but is not limited to, diabetes and smoking, and the use of medications including, but not limited to, prednisone or other steroids which are known to increase the likelihood of developing a bone healing disorder.

Because mature chondrocytes have little potential for replication, and since recruitment of other cell types is limited by the avascular nature of cartilage, mature cartilage has limited ability to repair itself. For this reason, transplantation of cartilage tissue or isolated chondrocytes into defective joints has been used therapeutically. However, tissue transplants from donors run the risk of graft rejection as well as possible transmission of infectious diseases. Although these risks can be minimized by using the patient's own tissue or cells, this procedure requires further surgery, creation of a new lesion in the patient's cartilage, and expensive culturing and growing of patient-specific cells. Better healing is achieved if the subchondral bone is penetrated, either by injury/disease or surgically, because the penetration into the vasculature allows recruitment and proliferation of undifferentiated cells to effect repair. Unfortunately, the biochemical and mechanical properties of this newly formed fibrocartilage differ from those of normal hyaline cartilage, resulting in inadequate or altered function. Fibrocartilage does not have the same durability and may not adhere correctly to the surrounding hyaline cartilage. For this reason, the newly synthesized fibrocartilage may be more prone to breakdown and loss than the original articular hyaline cartilage tissue.

Peptide growth factors are very significant regulators of cartilage growth and cartilage cell (chondrocyte) behavior (i.e., differentiation, migration, division, and matrix synthesis or breakdown). Chen et al., Am J. Orthop. 26:396-406 (1997). Growth factors that have been previously proposed to stimulate cartilage repair include, but are not limited to: i) insulin-like growth factor (IGF-1) (Osborn, J. Orthop. Res. 7:35-42 (1989) and Florini et al., J. Gerontol. 35: 23-30 (1980)); ii) basic fibroblast growth factor (bFGF) (Toolan et al., J. Biomec. Mat. Res. 41: 244-50 (1998) and Sah et al., Arch. Biochem. Biophys. 308:137-47 (1994)); iii) bone morphogenetic protein (BMP) (Sato et al., Clin. Orthop. Relat. Res. 183:180-187 (1984), Chin et al., Arthritis Rheum. 34: 314-24 (1991); and iv) transforming growth factor beta (TGF-β) (Hill et al., Prog. Growth Fac. Res. 4: 45-68 (1992), Gueme et al., J. Cell Physiol. 158: 476-84 (1994), and Van der Kraan et al., Ann. Rheum. Dis. 51: 643-47 (1992)). Treatment with peptide growth factors alone, or as part of an engineered device for implantation, could in theory be used to promote in vivo repair of damaged cartilage or to promote expansion of cells ex vivo prior to transplantation. However, because of their relatively small size, growth factors are rapidly absorbed and/or degraded, thus creating a great therapeutic challenge in trying to make them available to cells in vivo in sufficient quantity and for sufficient duration.

The data herein demonstrate what is believed to be the first demonstration that proliferating chondrocytes express a CysLT1 receptor. Further, the data herein demonstrate what is believed to be the first demonstration that cysteinyl leukotrienes reduce chondrocyte proliferation and that cysteinyl leukotrienes abrogate this reduction, thereby resulting in chondrocyte proliferation. Although it is not necessary to understand the mechanism of an invention, it is believed that chondrocyte proliferation may play a role in treating bone healing disorders, such as a non-union disease.

In one embodiment, the present invention contemplates a method comprising treating bone healing disorders by the administration of a CysLT1 receptor antagonist. In one embodiment, the receptor antagonist comprises montelukast sodium. In one embodiment, the receptor antagonist comprises montelukast sodium derivatives. In one embodiment, the receptor antagonist is administered orally. Although it is not necessary to understand the mechanism of an invention, it is believed that, mechanistically, the improved bone healing occurs via pharmacologic blockade of a chondrocyte CysLT1 receptor thereby leading to enhanced pre-hypertrophic chondrocyte proliferation. Consequently, some embodiments of the present invention contemplate that the CysLT1 receptor as a negative regulator of chondrocyte activity, which has important physiologic implications for fracture repair and other physiologic processes. The data shown herein demonstrates that chondrocyte CysLT1 receptor antagonist administration provides early and enhanced fracture repair, thereby decreasing bone fracture healing duration.

1. Chondrocyte Proliferation-Mediated Fracture Healing

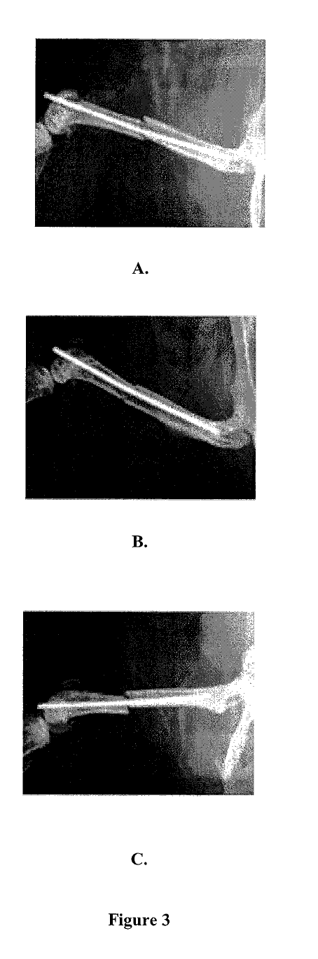

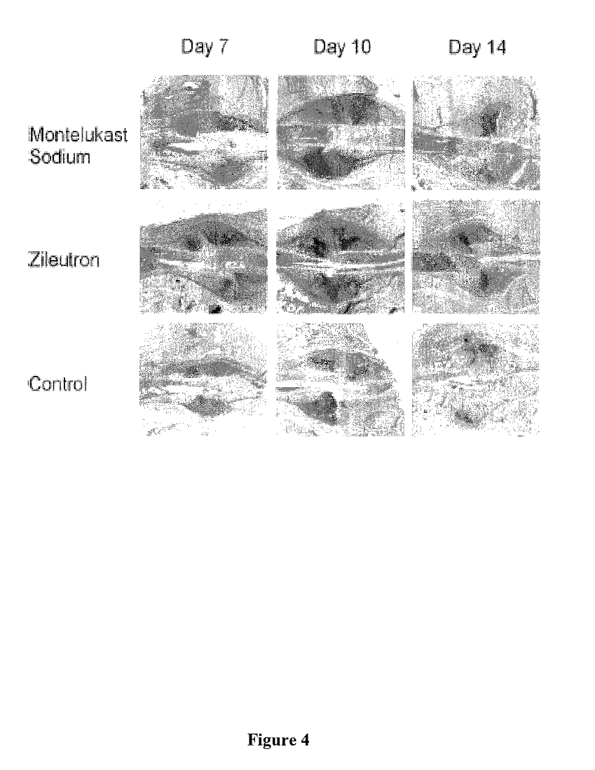

Because leukotrienes have been reported as potent inflammatory agents and may have a role as negative regulators of mesenchymal stem cell (MSC) differentiation, it was reasonable to determine as to whether inhibiting the effect of the cysteinyl leukotrienes would enhance chondrocyte proliferation in a bone healing from a fracture. See, Example II. Consequently, a mouse model for fracture repair was used to evaluate the effects of montelukast sodium, zileuton, or carrier at Days 7, 10, 14, and 21 post fracture. Day 0 post-fracture X-rays were taken while under anesthesia. See, FIG. 3. Subsequent X-rays were taken at Days 7, 10, 14, and 21 for all animals (N=4) until the time of sacrifice. Animals were treated with either montelukast sodium (1.5 mg/kg/Day), zileuton (45 mg/kg/Day), or control. Histology demonstrates that in treatments groups, enhanced chondroid formation was present as early as Day 7 and was sustained to Day 14, indicating rapid MSC differentiation and sustained chondrocyte proliferation. Histological sections for Days 7, 10, and 14 post-fracture where the differences between treated and control groups were most readily identifiable at early time points. See, FIG. 4. For example, evidence of healing was readily visible between the two treatment groups versus control as early as Day 7.

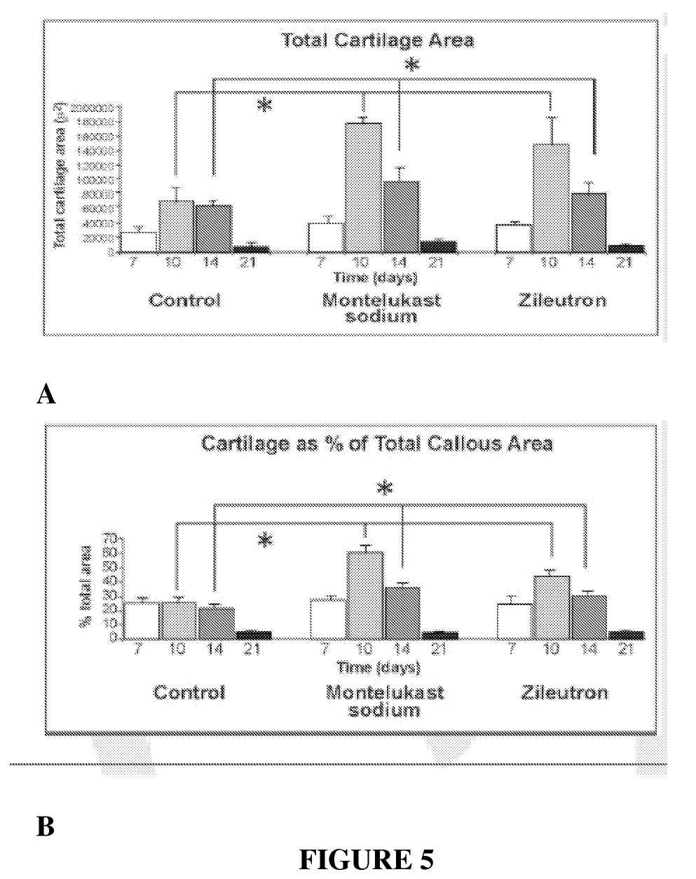

Quantitative measurements showed that both montelukast sodium and zileuton treatment resulted in a dramatically larger callous size with markedly increased amounts of unmineralized chondroid matrix at both Day 7 and Day 10. See, FIG. 5. This effect was restricted to the chondroid phase, and by Day 14 the endochondral process had progressed in all groups. In treatment groups, the larger initial callous resulted in a net increase of bone relative to control. Histomorphometry data demonstrate enhanced cartilage formation in both treatment groups when compared with controls. Both total cartilage area and cartilage area as a percent of total callous area are enhanced. See, FIG. 5A and FIG. 5B, respectively.

Consequently, quantification of total callous size, amount of chondroid, and percent chondroid were all significantly greater in both the montelukast sodium group and the zileuton group when compared to control, although changes were not significant when treatment with montelukast sodium was compared with zileuton. The results show that at Day 7 and Day 10 new bone formation was quantified using histomorphometry and was found to be significantly greater in the montelukast sodium and zileuton groups a compared with controls. By Day 21, animals in both montelukast sodium and zileuton groups had bridged the ends of the fracture and remodeled the callous to woven bone (data not shown). Non-unions, delayed unions, or acute infections were entirely absent

This data set reveals that enhanced early chondrogenesis is likely responsible for the larger callous size. The onset of bone formation, however, is not delayed, and new bone formation is clearly identifiable. This suggests that the endochondral process as a whole is enhanced, and that the increased chondroid phase does not occur at the expense of a delay in osteogenesis.

Taken together, these findings suggest that montelukast sodium and zileuton enhance chondrogenesis after an initial inflammatory response. Montelukast sodium is a specific CysLT1 receptor antagonist, while zileuton broadly blocks leukotriene synthesis. The similarities between montelukast sodium and zileuton suggest that cysteinyl leukotriene inhibition may provide a common mechanism for both drugs that initiate early changes in callous formation, thereby contributing to early fracture stabilization.

2. Chondrocyte CysLT1 Receptors

Until the present invention, CysLT1 receptors had not been identified within chondrocytes. Those in the art considered cysteinyl leukotriene effects to be limited to inflammatory responses, including those involved with bone trauma.

a. mRNA Expression

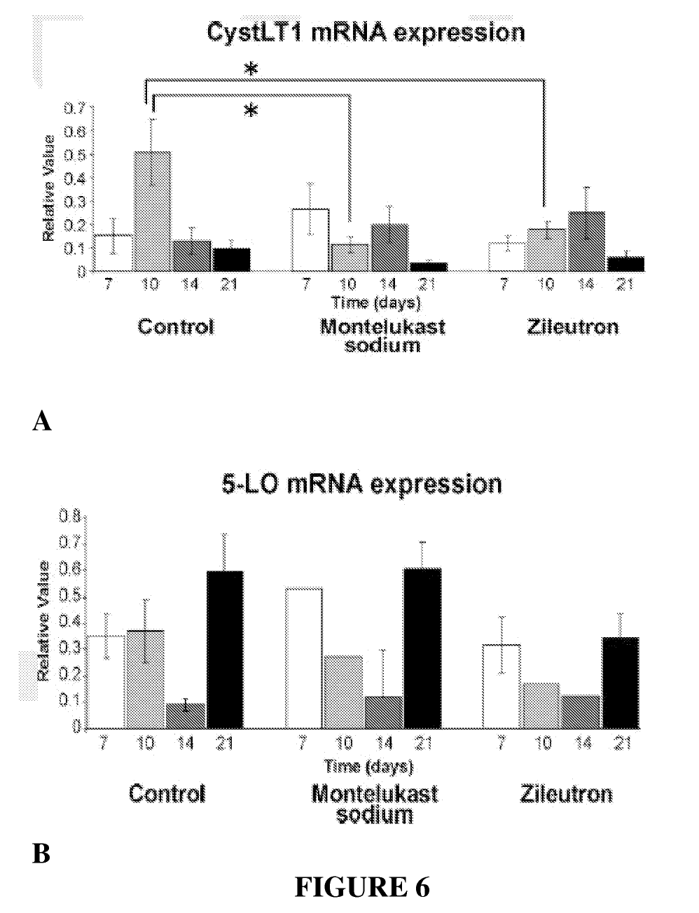

Levels of the 5-LO enzyme and the CysLT1 receptor were examined during the mouse model fracture repair studies detailed above. Gene expression analysis indicated that both CysLT1 receptor mRNA and 5-LO mRNA were expressed in fracture callous. See, FIG. 6A and FIG. 6B, respectively. In control animals, peak expression of the CysLT1 receptor occurred at Day 10, and was significantly higher than either the montelukast sodium or the zileuton treated groups. These gene expression data are consistent with our histology, particularly at Day 10, demonstrating an inverse relationship between callous size and CysLT1 expression. Specifically, low CysLT1 receptor levels observed in the montelukast and zileuton groups correspond with large callous sizes, supporting a role for the cysteinyl leukotrienes as negative regulators of chondrocyte proliferation.

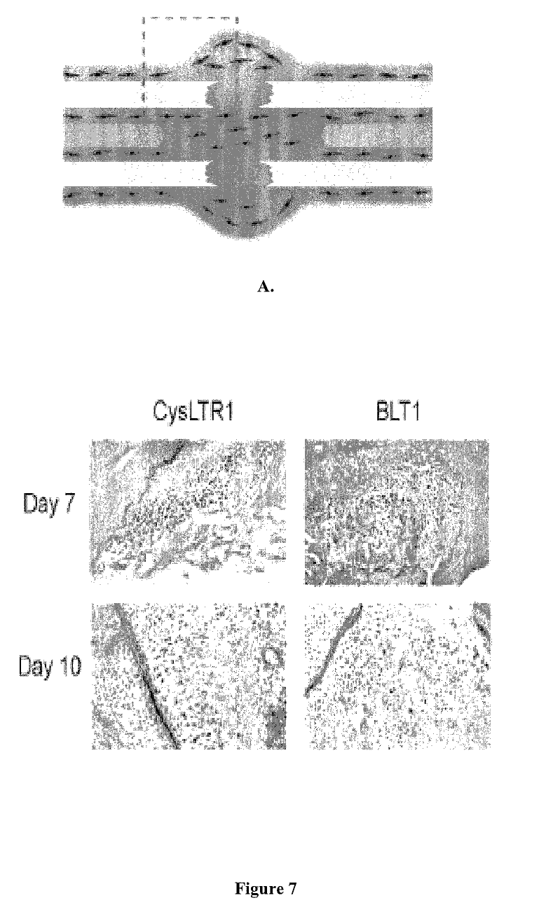

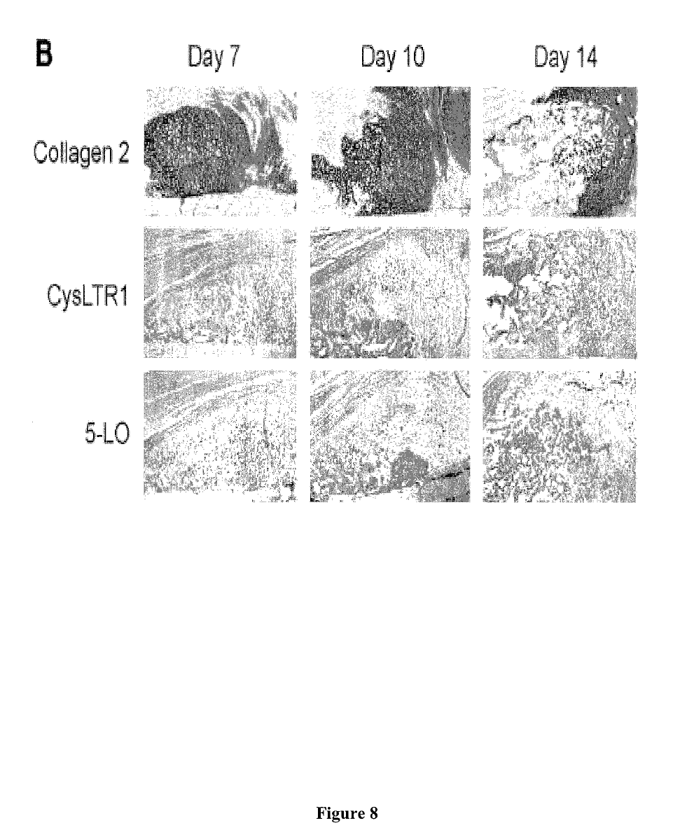

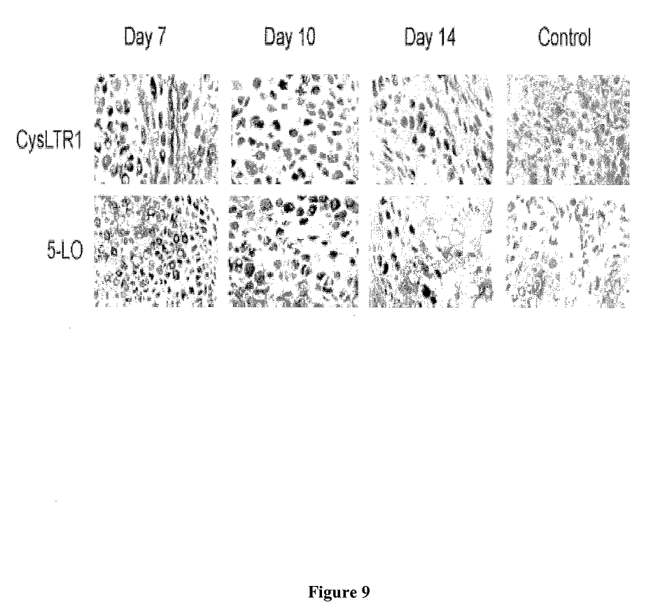

To elucidate the cells of origin which expressed the CysLT1 receptor, immunohistochemical staining on control sections was conducted. Control sections were chosen because peak expression was higher, and this also avoided potential regulatory effects of receptor expression in the presence of treatment drugs (i.e., for example, montelukast sodium or zileuton). Day 7 staining in the control sections was sparse, whereas staining was clearly present at Day 10, thereby showing expression in pre-hypertrophic chondrocytes. See, FIG. 7. It should be noted that neither immature chondrocytes nor hypertrophic chondrocytes expressed the CysLT1 receptor. The CysLT1 receptor and 5-lipoxygenase are expressed by chondrocytes early in the fracture repair process. Histologic sections were analyzed and show evidence of CysLT1 and 5-LO expression in chondrocytes. See, FIG. 8 and FIG. 9.

b. Cysteinyl Leukotriene Negative Regulation

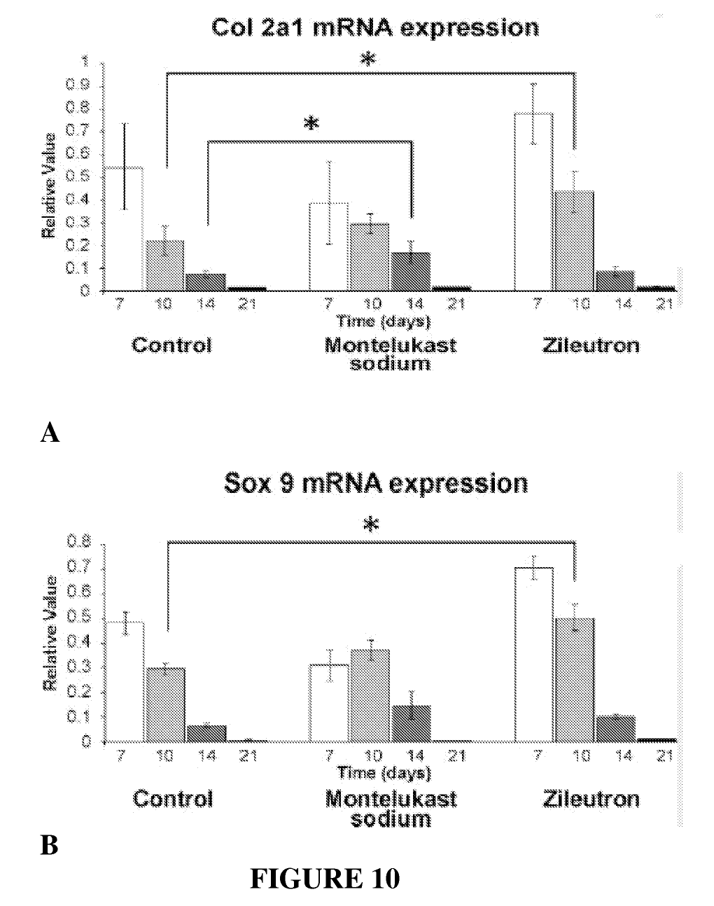

Given the unexpected finding of CysLT1 expression in fracture callous comprising a restricted expression in pre-hypertrophic chondrocytes, involvement of the endochondral process was ascertained. Fracture callous was harvested from animals (N=4) in the three treatment groups after sacrifice at days 7, 10, 14, and 21 and assayed for expression of chondrogenic markers by qPCR. See, FIG. 10. Peak levels of chondrocyte markers were seen in all groups at Day 7. These markers showed prolonged expression in treatment animals compared with controls. In control animals, expression drops markedly after Day 10. In the montelukast sodium group, Col 2a1 expression is elevated at Day 14 (P<0.05) and in the zileuton group both SOX9 and Col 2a1 are elevated at Day 10 (P<0.05). Although it is not necessary to understand the mechanism of an invention, it is believed that the expression of Sox9 and Col 2a mRNA indicate a cellular commitment to the chondrocyte phenotype. For example, mRNA expression of Sox9 and Col 2a in controls exhibits peak levels at day 7, when proliferating chondrocyte committed populations begin to expand. In the zileuton group, expression levels of both genes are increased significantly at Day 10. This is not seen in the montelukast sodium group; however, the Col 2a expression is elevated at Day 14 and Sox9 levels peak later than controls at Day 10.

c. Enhanced Chondrocyte Hypertrophy

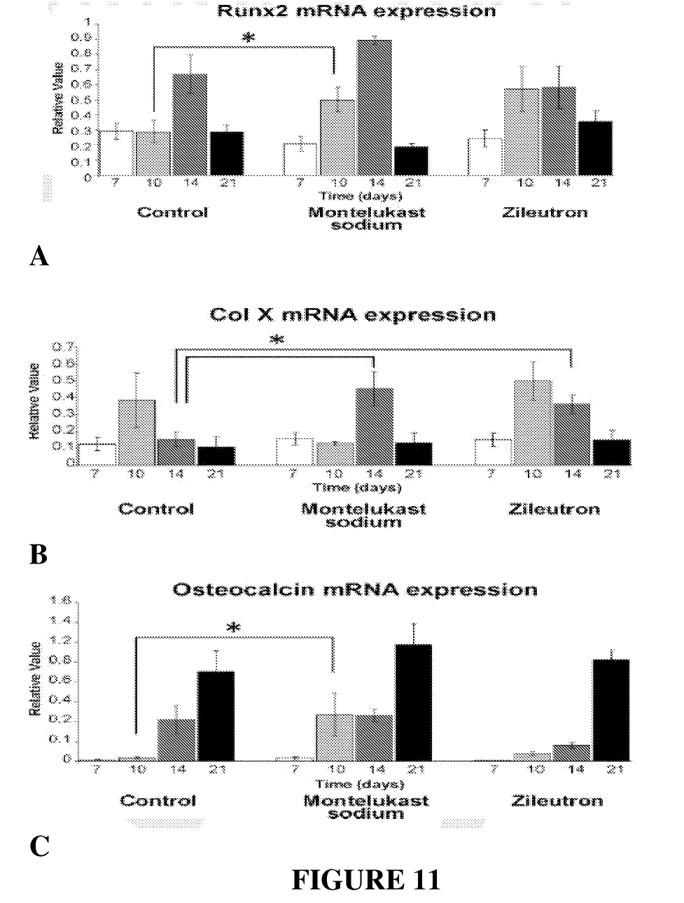

LTRA effects on endochondral ossification and early and sustained chondrogenesis prompted an investigation of the effects on osteogenesis. For example, Runx2 and Col 10a1 gene expression were also analyzed as markers of hypertrophic chondrocyte formation. Col 1 and osteocalcin levels were analyzed as markers of bone formation. See, FIG. 11A-C. In the montelukast sodium group, Runx2 mRNA levels are higher at Day 10 (P<0.05) and Col 10a1 mRNA levels are higher at Day 14 (P<0.01). In the zileuton group, Runx2 expression levels were increased at Day 10 and approached significance (P=0.06), while Col 10a1 expression was significantly higher (P<0.01) at Day 14. Early osteocalcin levels were also elevated in the montelukast sodium group (P<0.05) and zileuton group (P=0.07) at Day 10. Although it is not necessary to understand the mechanism of an invention, it is believed that the finding that Col 10a1 is elevated at Day 14 in the presence of montelukast sodium or zileuton likely reflects higher numbers of mature chondrocytes, a result of the sustained chondrocyte proliferation seen at earlier time points.

Further, montelukast sodium or zileuton decrease Col 1 mRNA levels at Day 7 as compared with controls, which suggests that some of the increase in chondrogenesis might occur at the expense of osteogenesis (data not shown). By Day 10, the Col 1 expression pattern and peaks are the same in all treatment groups at subsequent points. However, the increased expression of Runx2 in both montelukast sodium and zileuton treatment groups at Day 10 suggests an early transition to hypertrophic chondrocytes, suggesting that the callous is maturing more rapidly in the montelukast sodium and zileuton groups. A significantly elevated osteocalcin expression at Day 10 in the montelukast sodium group, wherein osteocalcin levels are approaching significance in the zileuton group (P=0.07) compared with controls, would also indicate that transition to osteogenesis occurs earlier in both treatment groups.

d. Differential Effects of Montelukast Sodium And Zileuton

While the overall effect of montelukast sodium and zileuton appear similar, one might suspect that both drugs interfere with CysLT1 receptor signaling. However, zileuton also blocks formation of the non-cysteinyl leukotrienes (i.e., for example, LTB4). The potential role of LTB4 in regulating chondrocyte proliferation was assessed using immunohistochemistry by localizing the BLT1 receptor, the main target for LTB4, in fracture callous.

The data demonstrated a strong chondrocyte-specific BLT1 expression. Immunohistochemical analysis of fracture callous taken from control animals at Day 10 shows mature chondroid tissue and early osseous response. Staining for the BLT1 receptor is restricted to chondrocytes. Notably, the BLT1 receptor expression pattern and timing differs from CysLT1 receptor expression pattern. Although it is not necessary to understand the mechanism of an invention, it is believed that such differences in gene expression patterns between BLT1 and CysLT1 when in the presence of montelukast sodium versus zileuton suggest a differential role for the BLT1 receptor. For example, on Day 7 BLT1 staining is primarily cytoplasmic, whereas by Day 10 BLT1 staining is both cytoplasmic and nuclear. See, FIG. 7B. Furthermore, histone gene expression by qRT-PCR is higher in the zileuton treated group, suggesting a potential role for BLT1 in regulating cell proliferation (data not shown).

### VI. LTRA Treatment of Trauma-Induced Bone Fractures

Recent studies have attempted to characterize trauma-induced bone fracture healing using pharmacogenetic models. Manigrasso et al., “Comparison of fracture healing among different inbred mouse strains” Calcif Tissue Int. 82:465-474 (2008). Quantitative trait locus analysis can be used to identify genes involved in biological processes. Healing of femur fractures was measured between C57BL/6, DBA/2, and C3H inbred strains of mice. In all strains, radiographic bridging of the fracture was apparent after 3 weeks of healing. Histology showed that healing occurred through endochondral ossification in all strains. Histomorphometric measurements found more bone in the C57BL/6 fracture calluses 7 and 10 Days after fracture. In contrast, more cartilage was present after 7 Days in the C3H callus, which rapidly declined to levels less than those of C57BL/6 or DBA/2 mice by 14 Days after fracture. An endochondral ossification index was calculated by multiplying the callus percent cartilage and bone areas as a measure of endochondral ossification. At 7 and 10 Days after fracture, the ossification index was highest in C57BL/6 mice. Using torsional mechanical testing, normalized structural and material properties of the C57BL/6 healing femurs were also higher than values from the DBA/2 or C3H mice 4 weeks after fracture. The data indicate that fracture healing proceeds more rapidly in C57BL/6 mice and demonstrate that genetic variability significantly contributes to the process of bone regeneration. Large enough differences exist between C57BL/6 and DBA/2 or C3H mice to permit a quantitative trait locus analysis to identify genes controlling bone regeneration.

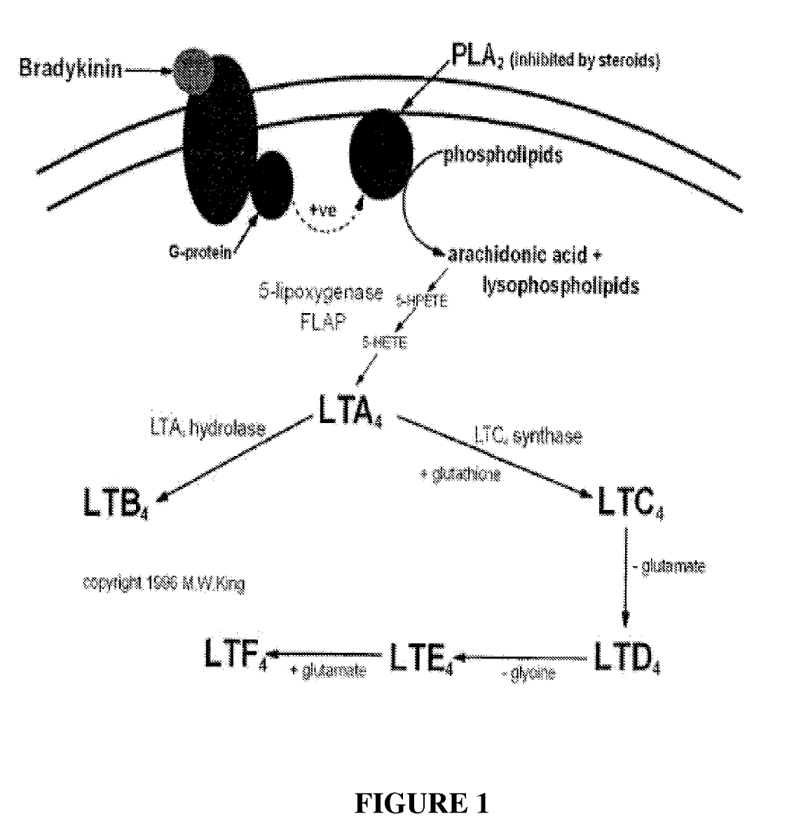

Recently, investigators have examined a potential role for leukotrienes in modulating trauma-induced fracture repair. A fracture study in knockout mice deficient in 5-lipoxygenase (5-LO) exhibited enhanced fracture repair, although the mechanism for this effect remained unclear. 5-LO functions for the leukotriene family in a manner analogous to cyclo-oxygenase in the prostaglandin family. O'Connor et al., “Methods For Bone Treatment By Modulating An Arachidonic Acid Metabolic Or Signaling Pathway” United States Patent Application Publication No. 2008/10280826, and FIG. 1. This enzyme, acting in concert with 5-lipoxygenase activating protein (FLAP), is believed to catalyze the conversion of arachidonic acid first to a series of intermediary metabolites, with an end result formation of two major groups of leukotrienes. For example, the formed leukotrienes may comprise an LTB4 leukotriene including, but not limited to, cysteinyl leukotrienes, LTC4, LTD4, LTE4, or LTF4.

While an earlier report suggested that 5-LO inhibition may enhance bone fracture repair, CysLT1 receptor antagonists were not disclosed. Simon et al., “Cyclo-oxygenase 2 function is essential for bone fracture healing” J Bone Miner Res. 17:963-976 (2002). In particular, 5-LO blockade is upstream of the CysLT1 receptor within the arachidonic acid metabolic pathway. Despite the molecular and histological similarities between fetal bone development and fracture healing, inflammation is an early phase of fracture healing that does not occur during development. Cyclo-oxygenase 2 (COX-2) is induced at inflammation sites and produces proinflammatory prostaglandins. To determine if COX-2 functions in fracture healing, rats were treated with COX-2-selective nonsteroidal anti-inflammatory drugs (NSAIDs) to stop COX-2-dependent prostaglandin production. Radiographic, histological, and mechanical testing determined that fracture healing failed in rats treated with COX-2-selective NSAIDs (celecoxib and rofecoxib). Normal fracture healing also failed in mice homozygous for a null mutation in the COX-2 gene. This shows that COX-2 activity is necessary for normal fracture healing and confirms that the effects of COX-2-selective NSAIDs on fracture healing is caused by inhibition of COX-2 activity and not from a drug side effect. Histological observations suggest that COX-2 is required for normal endochondral ossification during fracture healing. Because mice lacking Cox2 form normal skeletons, our observations indicate that fetal bone development and fracture healing are different and that COX-2 function is specifically essential for fracture healing.

### VII. Pharmaceutical Formulations

The present invention further provides pharmaceutical compositions comprising an LTRA described above. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral, intraarticular, or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing anti-inflammatory drugs, including but not limited to nonsteroidal anti-inflammatory drugs and corticosteroids may also be combined in compositions of the invention. Two or more combined compounds may be used together or sequentially.

Dosing is dependent on severity and responsiveness of the disease state to be treated, with the course of treatment lasting from several Days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compounds, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### EXPERIMENTAL

### Example II

### Animal Model for Bone Healing Disorder Studies

### Animals

C57B/6 mice were purchased (Charles River, Inc.Q3) and housed in the animal facility at the University of Massachusetts Medical School under IACUC approved protocol. Eight- to 9-week-old animals were used in the study.

### Fracture Technique

Institutional approval was obtained and all procedures were undertaken in accordance with approved IACUC methods. Animals were administered general anesthesia using IP injections of ketamine and xylazine. A midline skin incision over the knee joint was utilized and a median parapatellar arthrotomy was performed to expose the trochlear groove. A pilot hole was made using a 25 gauge needle to gain access to the femoral canal. The central cannula from a 22 gauge spinal needle was inserted into the canal and passed to the proximal femur in retrograde fashion. The wire was backed out slightly, cut, and reinserted. Wounds were closed, and the femur was held in a fixed position while a drop weight from a standard height was used to deliver a fixed traumatic injury to the mid portion of the femur, generating a fracture via three point bending, and ensuring that fractures were generated using a traumatic method with reproducible energy of injury (Marturano et al., 2008).

### Medications

Study medications included montelukast sodium (trade name Singulair), provided by the manufacturer (Merck, Inc.Q4) and zileuton (trade name Zyflo), purchased commercially (Sequoia, Inc.Q5). Medications were suspended in 1% methylcellulose (Sigma, Inc.Q6) and delivered by direct intragastric delivery. Montelukast sodium was delivered at a dose of 1.5 mg/kg/Day in a single dose, and zileuton was administered at 45 mg/kg/Day in divided doses. As the study medications are both currently FDA approved for the treatment of reactive airway disease, our dosing frequency was based on current prescription guidelines. Montelukast sodium was administered once daily by oral gavage. At the time the study began, zileuton was only approved for four times per Day dosing; as the drug needed to be administered by oral gavage and to maintain the normal sleep/awake cycle of the mouse colonies in the animal facilities, three doses of zileuton were dosed at 6-h intervals, with a 12-h break from 8 pm to 8 am. Control animals received 1% methylcellulose carrier only by a single daily oral gavage. The first dose of all medications was administered on post-fracture Day 1.

### Radiography

All animals were examined pre- and post-fracture using live fluoroscopy with an inverted Xiscan 1000 fluoroscope. Pre-fracture imaging was used to confirm correct positioning of the stabilizing wire, and post-fracture imaging was used to confirm correct fracture location and configuration. Additionally, standard radiographs were obtained in all animals immediately post-fracture with a high resolution MX-20 Faxitron on mammography film. Animals were anesthetized for additional X-rays to document fracture repair at 7, 14, 21, and 28 Days post-fracture. Animals were sacrificed immediately post-fracture if the fracture was not diaphyseal and transverse.

### Histology and Immunohistochemistry

Fracture specimens were harvested at various times (7, 10, 14, and 21 Days) and fixed in a solution of 4% paraformaldehyde, 0.1% CPC in PBS for 16 h at room temperature, and then embedded in paraffin after decalcification. Embedded tissues were sectioned into 6-mm slices, mounted on silane-coated glass slides (FisherQ7), de-paraffinized, and re-hydrated. Safranin-O: Slides were stained sequentially with Weigert's iron hematoxylin, fast green (FCF), and Safranin-O, then dehydrated sequentially in 95% ethyl alcohol, absolute ethyl alcohol, xylene and cover-slipped. Immunohistochemistry: Slides were washed in PBS. Non-specific tissue binding sites were blocked for 1 h at room temperature in 5% normal goat serum (NGS) (Santa CruzQ8) and incubated in a humidified chamber overnight at 48 C in 150 ml of diluted primary antibody per individual tissue section. Primary antibodies to CysLT1 and 5-LO were diluted (1:100 for all) in blocking solution. Following primary antibody incubation, sections were washed with PBS (33 min each) and visualized using an ABC biotin/avidin (Dako, Inc.Q9) amplification/reporter method using DAB as chromogen (brown ¼ positive identification). Slides were dried for 1 h at 378 C and cover-slipped using Pro-Texx (Learner Labs, Inc.Q10) mounting medium.

### Quantitative Real-Time PCR

Mice were sacrificed on Days 7, 10, 14, and 21 post-fracture. The fractured limb was carefully dissected free and all overlying tissue was carefully removed to expose the fracture callous. Callous tissue alone was then placed in TRIzol reagent, avoiding the underlying cortical bone. The tissue was ground using a Polytron homogenizer and total RNA was isolated as per the manufacturer's instructions (InvitrogenQ11). Any potential DNA contamination was removed by RNase-free DNase treatment. The reverse transcription reaction was performed on 1 mg of total RNA using the First Strand Synthesis Kit and random hexamer primers (Invitrogen). Relative transcript levels were measured by real-time PCR in a 25 ml reaction volume on 96-well plate using ABI PRISM 7000 FAST sequence detection system (Applied BiosystemsQ12), following the recommended protocol for SYBR-Green (Applied Biosystems). Transcript levels were normalized with 18S ribosomal RNA levels using primers from Applied Biosystems and SYBR-Green master mix (Applied Biosystems). The primers used for amplification are described in Table 2.

TABLE 2
XX^{Q13}X

| Gene | Forward | Reverse |
|---|---|---|
| Runx2 | 5′-CGG CCC TCC CTG AAC TCT-3′ | 5′-TGC CTG CCT GGG ATC TGT A-3′ |
| | SEQ ID NO: 1 | SEQ ID NO: 2 |
| Osteicalcin | 5′-CTG ACA AAG CCT TCA TGT CCA A-3′ | 5′-GCG CCG GAG TCT GTT CAC TA-3′ |
| | SEQ ID NO: 3 | SEQ ID NO: 4 |
| Col 10a1 | 5′-CCC AAG GAA AAG AAG CAC GTC-3′ | 5′-AGG TCA GCT GGA TAG CGA CAT C-3′ |
| | SEQ ID NO: 5 | SEQ ID NO: 6 |
| Collagen II | 5′-CTG GAA TGT CCT CTG CGA-3′ | 5′-TGA GGC AGT CTG GGT CTT CAC-3′ |
| | SEQ ID NO: 7 | SEQ ID NO: 8 |
| Collagen X | 5′-CCT GCA GCA AAG GAA AAC TC-3′ | 5′-TGT GGT AGT GGT GGA GGA CA-3′ |
| | SEQ ID NO: 9 | SEQ ID NO: 10 |
| Sox9 | 5′-GAG GCC ACG GAA CAG ACT CA-3′ | 5′-CAG CGC CTT GAA GAT AGC ATT-3′ |
| | SEQ ID NO: 11 | SEQ ID NO: 12 |
| CDK2 | 5′-ACA GCC GTG GAT ATC TGG AG-3′ | 5′-TTA GCA TGG TGC TGG GTA CA-3′ |
| | SEQ ID NO: 13 | SEQ ID NO: 14 |
| 5-LO | 5′-CCA TCA AGA GCA GGG AGA AG-3′ | 5′-ACC AGT CAT ACT GGC CGA AG-3′ |
| | SEQ ID NO: 15 | SEQ ID NO: 16 |
| CystLT1 | 5′-CAT CTT CCT GCT TTG GCT TC-3′ | 5′-ATT GCC AAA GAA ACC CAC AA-3′ |
| | SEQ ID NO: 17 | SEQ ID NO: 18 |
| Histone | 5′-CCAGCTGGTGTTTCAGATTACA-3′ | 5′-ACCCTTGCCTAGACCCTTTC-3′ |
| | SEQ ID NO: 19 | SEQ ID NO: 20 |

### Histomorphometry

Sectioning and histomorphometric measurements were performed in accordance with published methodologies (Gerstenfeld et al., 2005). Sagittal sections were reviewed and the most representative sections from the central portion of each specimen were chosen for analysis. The total callus area and cartilage area were measured in sections and quantified using imaging software.

### Statistical Methods

Student's t-test was performed to analyze the significance of the data for gene expression and histomorphometric analysis.

### REFERENCES

Anon. 1978. International nomenclature of constitutional diseases of bone. Revision, May, 1977. Am J Roentgenol 131:352-354.
Akino K, Mineda T, Mori N, Hirano A, Imaizumi T, Akita S. 2006. Attenuation of cysteinylleukotrienes induces human mesenchymal stem cell differentiation. Wound Repair Regen 14:343-349.
Beck A, Krischak G, Sorg T, Augat P, Farker K, Merkel U, Kinzl L, Claes L. 2003. Influence of diclofenac (group of nonsteroidal anti-inflammatory drugs) on fracture healing. ArchOrthop Trauma Surg 123:327-332.
Bergenstock M, Min W, Simon A M, Sabatino C, O'Connor J P. 2005. A comparison between the effects of acetaminophen and celecoxib on bone fracture healing in rats. J Orthop Trauma 19:717-723.
Boyce J A. 2008. Eicosanoids in asthma, allergic inflammation, and host defense. Curr Mol Med 8:335-349.
Denzlinger C, Rapp S, Hagmann W, Keppler D. 1985. Leukotrienes as mediators in tissue trauma. Science 230:330-332.
Einhorn T A. 2005. The science of fracture healing. J Orthop Trauma 19:S4-S6.
Engesaeter L B, Sudmann B, Sudmann E. 1992. Fracture healing in rats inhibited by locally administered indomethacin. Acta Orthop Scand 63:330-333.
Gerstenfeld L C, Wronski T J, Hollinger J O, Einhorn T A. 2005. Application of histomorphometric methods to the study of bone repair. J Bone Miner Res 20:1715-1722.
Gerstenfeld L C, Al-Ghawas M, Alkhiary Y M, Cullinane D M, Krall E A, Fitch J L, Webb E G, Thiede M A, Einhorn T A. 2007. Selective and nonselective cyclooxygenase-2 inhibitors and experimental fracture-healing. Reversibility of effects after short-term treatment. J Bone Joint Surg Am 89:114-125.
Hogevold H E, Grogaard B, Reikeras 0.1992. Effects of short-term treatment with corticosteroids and indomethacin on bone healing. A mechanical study of osteotomies in rats. Acta Orthop Scand 63:607-611.
Keller J, Bayer-Kristensen I, Bak B, Bunger C, Kjaersgaard-Andersen P, Lucht U, Melsen F. 1989. Indomethacin and bone remodeling. Effect on cortical bone after osteotomy in rabbits. Acta Orthop Scand 60:119-121.
Manigrasso M B, O'Connor J P. 2006. Accelerating fracture healing by manipulating arachidonic acid metabolism. Chicago Q14, IL. p 0070.
Marturano J E, Cleveland B C, Byrne M A, O'Connell S L, Wixted J J, Billiar K L. 2008. An improved murine femur fracture device for bone healing studies. J Biomech 41:1222-1228.
Murnaghan M, Li G, Marsh D R. 2006. Nonsteroidal anti-inflammatory drug-induced fracture nonunion: An inhibition of angiogenesis? J Bone Joint Surg Am 88:140-147.
Schindeler A, McDonald M M, Bokko P, Little D G. 2008. Bone remodeling during fracture repair: The cellular picture. Semin Cell Dev Biol 19:459-466.
Simon A M, O'Connor J P. 2007. Dose and time-dependent effects of cyclooxygenase-2 inhibition on fracture-healing. J Bone Joint Surg Am 89:500-511.
Sudmann E, Dregelid E, Bessesen A, Morland J. 1979. Inhibition of fracture healing by indomethacin in rats. Eur J Clin Invest 9:333-339.
Traianedes K, Dallas M R, Garrett I R, Mundy G R, Bonewald L F. 1998. 5-Lipoxygenase metabolites inhibit bone formation in vitro. Endocrinology 139:3178-3184.

We claim:

1. A method, comprising:
a) providing;
i) a patient comprising at least one symptom of a bone healing disorder;
ii) a composition comprising a cysteinyl-leukotriene receptor antagonist capable of reducing said symptom;
b) administering said receptor antagonist to said patient under conditions such that said symptom is reduced.

2. The method of claim 1, wherein said bone healing disorder is selected from the group consisting of, non-union predisposition, non-healing non-union fractures, osteopenia, osteogenesis imperfecta, critical size defects, non-critical size defects, osteochondral defects, subchondral defects, and osteochondritis dessicans.

3. The method of claim 1, wherein said patient further comprises a chondrocyte, wherein said chondrocyte expresses at least one cysteinyl leukotriene-1 receptor.

4. The method of claim 1, wherein said administering of said receptor antagonist stimulates said chondrocyte to proliferate.

5. The method of claim 1, wherein said receptor antagonist comprises montelukast.

6. The method of claim 1, wherein said receptor antagonist comprises a montelukast derivative.

7. The method of claim 1, wherein said administering is parenteral.

8. The method of claim 1, wherein said administering is oral.

9. The method of claim 1, wherein said administering is intraarticular.

10. The method of claim 1, wherein said bone disorder is caused by a disease.

11. The method of claim 1, wherein said bone disorder is congenital.
